SEQUENCE LISTING (14 sequences)

SEQ ID NO: 1 - DNA, 6740 bp, Artificial Sequence
  Synthetic
ctggcgtaatagcgaagaggcccgcaccgatcgcccttcccaacagttgcgcagcctgaatggcgaatggcgcttcgcttggtaagttaactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtaatgtgagttagctcactcattaggcaccccaggctttacactttatgcttccggctcgtatgttgtgtggaattgtgagcggataacaatttcacacaggaaacagctatgaccatgattacgccaagctacgtaatacgactcactataggggcccgtgcaattgaagccggctggcgccaagcttctctgcaggatatctggatccttgaaaatggagtgagctgagttaatgatgaccgacttcccactgagggcctctagaatgttcaacactatggctctattatgtgcattgatgtatgtaaaattgttcgtatttttccttttttcttgaaaatgtttgaggaacacctttatatttttgaattttagattctttgagcctaatcaaataaacaaaaggatttccactcttgtttaccgaaagctttatataggctattgcccaaaaatgtatcgccaatcacctaatttggagggatgaacatggaaatagaggagattatagagaaagttgctaggggagagatcaagtttcatcaagtagaaaactatgtgaatggggataaaaggcttgcaactgagataagaaggagagccttggagaaaaaacttggaatacagctaaagcacattggccactactcaattgatccaaacgaagttattggaaggaacattgaaaacatgataggtgtcgttcaaatacccatgggtattgcgggtcctctaaagattaatggtgaatatgcaaaaggtgagttttacattcccctagccacaactgaaggggccttggttgcttcagtaaacagaggttgttctgctctaacagaggctggtggagtttacacaaccctaatagatgacaagatgactagagctcctctattgaaatgtccaaacgccagaagggcgagagaagtagctgaatgggtcaagaataatctggattacctccaagaaaaagctgttagtaaggttactcgtcacggaaagcttaggggggtaaagccttttatcgttgggagaaacttatacttaagatttgaattcgaaactggagatgccatgggaatgaacatggttacaatagcgagcgaagagataatgaaagtaatagaagaagaattccccgatgtaaaatatttggctctttctggtaatctgtgcgtagataagaagccaaacgccttaaacttcatcctgggaagaggaaagaccattattgctgaagctgtagttcctagagagattgttaagaaaaagctaaagaccactccagagcttatcgctgaagtaaactacctaaagaacttagtgggttcggcccaagcaggttcctacggttttaacgctcactttgccaacattgtaggggcgatattcctagccacaggtcaggatgaggctcaaattacggaaggtgcacatggaataaccctcgctgaagtcactgaagatggagatctatacataagcataacaatgccgagcctagaaatagggacggttggaggtgggactagagtacctcctcaaagagaagctctagaaattatgggggttgctggaggaggagatcctccagggatgaatgctaagaaatttgcggagatagttgctggtgcggttttggctggtgagctctctcttctggccgcaattgccgccaaacatttggctagggctcataaaatgcttgggagatgaggatccacgaattcgctagcttcggccgtgacgcgtctccggatgtacaggcatgcgtcgaccctctagtcaaggcggggggcgaggcgaggggggagaggagagagtgtggggtgcggtgccaaaatgacacccaaacttacaataaagttggaaggtgtcatacaggcacacccccatcaggaggaggaataagccgaataatggtaatagaacggtgcctatcactaaagacaaggacatcagagagatgattatcataaagataatcgacaaactccctgaaggagacatcctcaagctcaacatagaaattgtaattgacaatagacttaccaagagcggtgaacaaagtttccaaaatatgaataacacgctcaagagtgagattatcgttagataacaactcctccgccttctcaagggcacgctcaaagagatactcgaaaccacgctccttaagttctgaaagctcatccttcaaatactcaagacgctcaaggagagccctcttaagtgaatcactcatcttattaccattcacagacaaatcaaactcaatacggctaatgaactcttcaagctcctgaacacgcttctctacgaggtgagagcaagacatagaaagataacgtttaatgttagtgaggaaaccataacgttcagtcctattactatactcgaagacgaaacggacaaaatcccaatcggacacggagctctcatcaaaattagactgctcaaagtaattaacaaaattgacgaaaagcttcctcgacgcatacttgagctcgaagaagacctgaggaacatcaagaggcaaagagtaataattatcaccagcccaaacatcaaaatcctttgacttagtatcctcagagagaagctcaccgaaataggacagcaacacgttcttccaaatatcacgcaatttctttagatcactctcgctgagaagaggattaagcctaaaccatttggttgaagacttatcatagcaaatgaaagtaacgatagcatcaatatggaagtgaggctcaaaggggttcttatcaccagtaacatgaacattgatagtaaaaccaaataggagattaccagagatatgttccttggaagcaaggtatgataaaaactccttgatggctttagcaccagcatccttgaaagccctaaacaatgaactatcccccttcttgagagaagcccaaatactgaaactaacgtccttaggagcggtaaggacaaaatgacggacaggaataaggtgatgaatagtttcaacccccccactcctgtagctaacaagagaaccctcaagaagttcactaagcacaagcaaatccctaagaaccctcttagcctcctgagaacccttggagatacccttcacaggatgatacttaccagcgtgaacctttgaaatataagcaatacggaacttatgagaactattatcatcaagaaacacctcaagaacaccactattcacgaataggtgaagaagaagcgtatcacttgagaacccgagcttttggattaactttgcgtaatcttggggaacctcaacaaagaacctatccgcataaagagtaagagacttggaaatagaacctatgagagcacgagcaaaatcaagatacttacgcttgcgagagagaagctttgctctctccctatcatcaaggaaaagcctcgataactgcttgtttatagagcggagttctttggagattttcctgagcttaaggacagttctttcaacttctacaaaaccagagtgctcaagccttgaaatgactgaacgatacttatcaaaccagataaaactactgccatcaccctcagatgacttagaactgacaccacacaccttcaatagagggcttttactctcagtatttgaagagaactcttctgaaattccttcattggttgtggatatatcaagataaacccaacgggggcggggtcgagaaccagatgacccacccgagatgaaagtgtcgagggtaagggttggatgaacctcattacatagaataggctggtcatcatagctaaggtgcccaacccccaacccatcaagcaaagagtttttaaactttgaagtgtatatcaccattgcaggggcctcctatggtttcctattgtcttgggggtgtttagggggagctccccactaaggggggctcccccaatacccaacgcgtttctaaaggataatattctcaaggatttataactcctacggagtataattgaggtagtaaacatcgttctccaccttcgtatgctttgaaacacgcacaataccaaacctctcacgaggataactaacgagagacgaagacttcttcttagacttacgagtcctcctcgaattaagattttcaacgtaactatcaatggcatcaagatcagcatcaacctcatacgtccaattaccaatacgaacaatgagcttcccgctatgagggtcatagcgatattcaccaaccctatcaacctcagaagcgggaacatagaagaaaccatgctcacggagaaaatagaacatgcgccgagcaacccgataccccccaaacacattgtgaataatcctaccagcacgataatacata

SEQ ID NO: 2 - DNA, 7825 bp, Artificial Sequence
  Synthetic
ctggcgtaatagcgaagaggcccgcaccgatcgcccttcccaacagttgcgcagcctgaatggcgaatggcgcttcgcttggtaagttaactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtaatgtgagttagctcactcattaggcaccccaggctttacactttatgcttccggctcgtatgttgtgtggaattgtgagcggataacaatttcacacaggaaacagctatgaccatgattacgccaagctacgtaatacgactcactataggggcccgtgcaattgaagccggctggcgccaagcttctctgcaggatatctccttaacatttctccaaaatatttcggatttgtttaatatttaactgagatttaatttacaaatacgacaatataatctcacaaagataaaagttttatagaaaattttcttcatcattctgggtgataaatgtttatcaaagtaaagtattaggaaaaatgttaacataagtttgtaaaattctggaggtgaaaaaatggccggaattgaagttcagattcttgaaaaaaaggaagattctattaagtttgtcttaaagggggttcatgtttcgtttgcaaatgcactaagaaggacaattttaggggaagtcccaacatttgcagttgatgaagtagaattttatgaaaacgactcagcactttttgatgaaatcattgcccacagattagcaatgattccactaactactccagttgataggtttgagttggacgcattagaacttgatgattacacggtaactctctctctagaagctgaaggcccagggattgtatattcaggtgatttaaagagtgatgatccggatgtaaaacctgtaaatccaaacattccaattgtaaagctcgctgagggacaaagacttgtatttaatgcctatgccaagcttggtagaggaaaggatcatgctaagtggcaacctggatttgtgtattacaagtactacactatcgttcatatcagcaagtcaatccccgaatggaaggagctgaaaaaacttgcaaagaaaaggggtcttcctgtggaagaaactgaggaagaagtactagtgacaacaataaaacctttctacataccaaaggatttcgaagagtatgaaggtaaggaaatctgggaggagatagtgccaaatacttacatatttacagttgaaacaaacggagagctccccgtagaggaaatagtatccatagctcttaaaatcttgatgagaaaggccgataggtttataagtgaacttcaaaaattgacctctcatcaccatcaccatcactgaaatcttttttagcacttttctttttccttaaattttcgcaaataatttttaactatgaggcttatctttttctagggtgatatcggatccttgaaaatggagtgagctgagttaatgatgaccgacttcccactgagggcctctagaatgttcaacactatggctctattatgtgcattgatgtatgtaaaattgttcgtatttttccttttttcttgaaaatgtttgaggaacacctttatatttttgaattttagattctttgagcctaatcaaataaacaaaaggatttccactcttgtttaccgaaagctttatataggctattgcccaaaaatgtatcgccaatcacctaatttggagggatgaacatggaaatagaggagattatagagaaagttgctaggggagagatcaagtttcatcaagtagaaaactatgtgaatggggataaaaggcttgcaactgagataagaaggagagccttggagaaaaaacttggaatacagctaaagcacattggccactactcaattgatccaaacgaagttattggaaggaacattgaaaacatgataggtgtcgttcaaatacccatgggtattgcgggtcctctaaagattaatggtgaatatgcaaaaggtgagttttacattcccctagccacaactgaaggggccttggttgcttcagtaaacagaggttgttctgctctaacagaggctggtggagtttacacaaccctaatagatgacaagatgactagagctcctctattgaaatgtccaaacgccagaagggcgagagaagtagctgaatgggtcaagaataatctggattacctccaagaaaaagctgttagtaaggttactcgtcacggaaagcttaggggggtaaagccttttatcgttgggagaaacttatacttaagatttgaattcgaaactggagatgccatgggaatgaacatggttacaatagcgagcgaagagataatgaaagtaatagaagaagaattccccgatgtaaaatatttggctctttctggtaatctgtgcgtagataagaagccaaacgccttaaacttcatcctgggaagaggaaagaccattattgctgaagctgtagttcctagagagattgttaagaaaaagctaaagaccactccagagcttatcgctgaagtaaactacctaaagaacttagtgggttcggcccaagcaggttcctacggttttaacgctcactttgccaacattgtaggggcgatattcctagccacaggtcaggatgaggctcaaattacggaaggtgcacatggaataaccctcgctgaagtcactgaagatggagatctatacataagcataacaatgccgagcctagaaatagggacggttggaggtgggactagagtacctcctcaaagagaagctctagaaattatgggggttgctggaggaggagatcctccagggatgaatgctaagaaatttgcggagatagttgctggtgcggttttggctggtgagctctctcttctggccgcaattgccgccaaacatttggctagggctcataaaatgcttgggagatgaggatccacgaattcgctagcttcggccgtgacgcgtctccggatgtacaggcatgcgtcgaccctctagtcaaggcggggggcgaggcgaggggggagaggagagagtgtggggtgcggtgccaaaatgacacccaaacttacaataaagttggaaggtgtcatacaggcacacccccatcaggaggaggaataagccgaataatggtaatagaacggtgcctatcactaaagacaaggacatcagagagatgattatcataaagataatcgacaaactccctgaaggagacatcctcaagctcaacatagaaattgtaattgacaatagacttaccaagagcggtgaacaaagtttccaaaatatgaataacacgctcaagagtgagattatcgttagataacaactcctccgccttctcaagggcacgctcaaagagatactcgaaaccacgctccttaagttctgaaagctcatccttcaaatactcaagacgctcaaggagagccctcttaagtgaatcactcatcttattaccattcacagacaaatcaaactcaatacggctaatgaactcttcaagctcctgaacacgcttctctacgaggtgagagcaagacatagaaagataacgtttaatgttagtgaggaaaccataacgttcagtcctattactatactcgaagacgaaacggacaaaatcccaatcggacacggagctctcatcaaaattagactgctcaaagtaattaacaaaattgacgaaaagcttcctcgacgcatacttgagctcgaagaagacctgaggaacatcaagaggcaaagagtaataattatcaccagcccaaacatcaaaatcctttgacttagtatcctcagagagaagctcaccgaaataggacagcaacacgttcttccaaatatcacgcaatttctttagatcactctcgctgagaagaggattaagcctaaaccatttggttgaagacttatcatagcaaatgaaagtaacgatagcatcaatatggaagtgaggctcaaaggggttcttatcaccagtaacatgaacattgatagtaaaaccaaataggagattaccagagatatgttccttggaagcaaggtatgataaaaactccttgatggctttagcaccagcatccttgaaagccctaaacaatgaactatcccccttcttgagagaagcccaaatactgaaactaacgtccttaggagcggtaaggacaaaatgacggacaggaataaggtgatgaatagtttcaacccccccactcctgtagctaacaagagaaccctcaagaagttcactaagcacaagcaaatccctaagaaccctcttagcctcctgagaacccttggagatacccttcacaggatgatacttaccagcgtgaacctttgaaatataagcaatacggaacttatgagaactattatcatcaagaaacacctcaagaacaccactattcacgaataggtgaagaagaagcgtatcacttgagaacccgagcttttggattaactttgcgtaatcttggggaacctcaacaaagaacctatccgcataaagagtaagagacttggaaatagaacctatgagagcacgagcaaaatcaagatacttacgcttgcgagagagaagctttgctctctccctatcatcaaggaaaagcctcgataactgcttgtttatagagcggagttctttggagattttcctgagcttaaggacagttctttcaacttctacaaaaccagagtgctcaagccttgaaatgactgaacgatacttatcaaaccagataaaactactgccatcaccctcagatgacttagaactgacaccacacaccttcaatagagggcttttactctcagtatttgaagagaactcttctgaaattccttcattggttgtggatatatcaagataaacccaacgggggcggggtcgagaaccagatgacccacccgagatgaaagtgtcgagggtaagggttggatgaacctcattacatagaataggctggtcatcatagctaaggtgcccaacccccaacccatcaagcaaagagtttttaaactttgaagtgtatatcaccattgcaggggcctcctatggtttcctattgtcttgggggtgtttagggggagctccccactaaggggggctcccccaatacccaacgcgtttctaaaggataatattctcaaggatttataactcctacggagtataattgaggtagtaaacatcgttctccaccttcgtatgctttgaaacacgcacaataccaaacctctcacgaggataactaacgagagacgaagacttcttcttagacttacgagtcctcctcgaattaagattttcaacgtaactatcaatggcatcaagatcagcatcaacctcatacgtccaattaccaatacgaacaatgagcttcccgctatgagggtcatagcgatattcaccaaccctatcaacctcagaagcgggaacatagaagaaaccatgctcacggagaaaatagaacatgcgccgagcaacccgataccccccaaacacattgtgaataatcctaccagcacgataatacata

SEQ ID NO: 3 - DNA, 24 bp, Artificial Sequence
  Synthetic
atggaaatagaggagattatagag

SEQ ID NO: 4 - DNA, 36 bp, Artificial Sequence
  Synthetic
atcatcggatcctcatctcccaagcattttatgagc

SEQ ID NO: 5 - DNA, 37 bp, Artificial Sequence
  Synthetic
ctcctctatttccatgttcatccctccaaattaggtg

SEQ ID NO: 6 - DNA, 33 bp, Artificial Sequence
  Synthetic
ggaaccggatccttgaaaatggagtgagctgag

SEQ ID NO: 7 - DNA, 31 bp, Artificial Sequence
  Synthetic
ctattagatatctccttaacatttctccaaa

SEQ ID NO: 8 - DNA, 38 bp, Artificial Sequence
  Synthetic
ctgaacttcaattccggccattttttcacctccagaat

SEQ ID NO: 9 - DNA, 31 bp, Artificial Sequence
  Synthetic
aaatggccggaattgaagttcagattcttga

SEQ ID NO: 10 - DNA, 41 bp, Artificial Sequence
  Synthetic
gtgatggtgatggtgatgagaggtcaatttttgaagttcac

SEQ ID NO: 11 - DNA, 38 bp, Artificial Sequence
  Synthetic
catcaccatcaccatcactgaaatcttttttagcactt

SEQ ID NO: 12 - DNA, 31 bp, Artificial Sequence
  Synthetic
tcaattgatatcaccctagaaaaagataagc

SEQ ID NO: 13 - DNA, 22 bp, Artificial Sequence
  Synthetic
ccaacatttgcagttgatgaag

SEQ ID NO: 14 - DNA, 25 bp, Artificial Sequence
  Synthetic
ctcttcgaaatcctttggtatgtag

(19) United States
(12) Patent Application Publication
(10) Pub. No.: US 2011/0262954 A1
(43) Pub. Date: Oct. 27, 2011

(54) SHUTTLE VECTOR BASED TRANSFORMATION SYSTEM FOR PYROCOCCUS FURIOSUS

(75) Inventors: Michael Thomm, Regensburg (DE); Winfried Hausner, Regensburg (DE); Ingrid Waege, Regensburg (DE)

(73) Assignee: UNIVERSITAT REGENSBURG, Regensburg (DE)

(21) Appl. No.: 13/048,329

(22) Filed: Mar. 15, 2011

Related U.S. Application Data
(60) Provisional application No. 61/315,178, filed on Mar. 18, 2010.
(60) Provisional application No. 61/379,601, filed on Sep. 2, 2010.

(51) Int. Cl. C12P 1/00; C12N 1/00; C12Q 1/02; C12N 15/74; C12N 9/02; C12N 15/63; C12N 9/12

(52) U.S. Cl. 435/29; 435/320.1; 435/243; 435/194; 435/471; 435/189; 435/41

(57) ABSTRACT

The present invention relates to vectors for transforming archaea and to transformed archaea, and in particular to shuttle vector systems for transformation of members of the genus Pyrococcus.

### CROSS-REFERENCE TO RELATED APPLICATIONS

This application claims the benefit of U.S. Prov. Appl. 61/315,178, filed Mar. 18, 2010 and U.S. Prov. Appl. 61/379, 601, filed Sep. 2, 2010, each of which the entire contents are incorporated herein by reference.

### FIELD OF THE INVENTION

The present invention relates to vectors for transforming archaea and to transformed archaea, and in particular to shuttle vector systems for transformation of members of the genus Pyrococcus.

### BACKGROUND OF THE INVENTION

Several reports addressed the initial establishment of genetic techniques for the Thermococcales, a major order of hyperthermophilic euryarchaeota including the genera Thermococcus and Pyrococcus. The first experiments described used the plasmid pGT5 from Pyrococcus abyssi. This plasmid is only 3440 by in size and replicates via a rolling circle mechanism (7). The archaeal plasmid was fused with a pUC 19 vector to create a potential shuttle vector between Escherichia coli and Pyrococcus furiosus (1). This construct could be transformed in both organisms by CaCl₂ treatment. Later, this construct was modified by introducing the alcohol dehydrogenase gene from Sulfolobus solfataricus as a selectable marker (3). The resulting plasmids pAG1 and pAG2 were maintained for several generations in E. coli, in the euryarchaeote P. furiosus and also in the crenarchaeote S. acidocaldarius. The presence of these plasmids in the two archaea conferred resistance to butanol and benzyl alcohol.

As the attempts to use this selection system for P. abyssi failed, a new shuttle vector, pYS2, was created (17). This construct is also based on the archaeal pGT5 plasmid and a bacterial vector, pLitmus38. It contains the pyrE gene of S. acidocaldarius, a key enzyme of the pyrimidine biosynthetic pathway, as a selectable marker. For the transformation procedure a Pyrococcus strain was used containing a pyrE mutation which led to a uracil-auxotrophic phenotype. Using the shuttle vector pYS2 in combination with a polyethylene glycol-spheroplast method, it was possible to transform the pyrE mutant of P. abyssi to uracil prototrophy. Although the transformation frequency was very low, the shuttle vector was stably maintained at high copy number under selective conditions in both E. coli and P. abyssi (17).

A major breakthrough in the establishment of genetic tools for hyperthermophilic euryarchaeota was the development of a targeted gene disruption system by homologous recombination in Thermococcus kodakaraensis KOD1 (23). A uracil-auxotrophic strain was converted with a disruption vector harboring the pyrF marker within the trpE gene to a uracil-prototrophic and a tryptophan-auxotrophic strain by double-crossover recombination. Due to the natural competence for DNA uptake, the high transformation efficiency and the high incorporation rate of DNA into its genome by homologous recombination, the system led to the identification of novel biochemical pathways, discovery of new enzyme functions and further elucidation of proteins involved in the basic process of transcription (4, 11, 20, 22).

A further improvement of this genetic system was the discovery that overexpression of the 3-hydroxy-3-methylglutaryl coenzyme A (HMG-CoA) reductase gene is connected with the resistance against the antibiotic simvastatin (18). This selection system was first described in halophiles (15) and has the great advantage that there is no need for a certain host strain with a particular defect or auxotrophy toward an amino acid (18).

Despite these finding, useful genetic techniques and tools for members of the genus Pyrococcus, a major model organism of hyperthermophilic archaea have not been developed. What is needed in the art are improved genetic tools that are useful with the genus Pyrococcus.

### SUMMARY OF THE INVENTION

The present invention relates to vectors for transforming archaea and to transformed archaea, and in particular to shuttle vector systems for transformation of members of the genus Pyrococcus.

Accordingly, in some embodiments, the present invention provides a shuttle vector comprising, in operable association: a bacterial replication origin, a bacterial selection marker gene, a rolling circle replication initiator protein gene, an antibiotic resistance gene that confers selectability in an archaeon, and a regulated archaeal promoter 5′ to a cloning site. The present invention is not limited to the use? of any particular regulated promoter. In some embodiments, the regulated promoter comprises a glutamate dehydrogenase gene (gdh) promotor, fructose-1, 6 bisphosphatase (fbp) promoter, or archaeal heat shock promoter. In some embodiments, the promoter includes regulatory elements that allow regulated expression. Examples of such regulatory elements include, but are not limited to, TrmB and TrmB-like regulatory factor response elements. The present invention is not limited to any particular rolling circle replication initiator protein gene. In some embodiments, the rolling circle replication initiator protein gene is selected from the group consisting of Rep74 and Rep75. The present invention is not limited to the use of any particular antibiotic resistance gene. In some embodiments, the antibiotic resistance gene that confers selectability in an archaea is hmg-CoA reductase. The present invention is not limited to the use of any particular origin of replication. In some embodiments, the bacterial replication origin is oriC. In some embodiments, the bacterial selection marker gene is different than said antibiotic resistance gene that confers selectability in an archaea. The present invention is not limited to the use of any particular bacterial selection marker gene. In some embodiments, the bacterial selection marker gene is selected from the group consisting of an auxotrophic marker gene and a gene that confers antibiotic resistance on a host cell. The present invention is not limited to the use of any particular antibiotic resistance. In some embodiments, the antibiotic is selected from the group consisting of kanamycin, ampicillin, tetracycline, Zeocin, neomycin, chloramphenicol and hygromycin. The present invention is not limited to any particular auxotrophic marker. In some embodiments, the auxotrophic marker is a gene selected from the group consisting of LEU2 gene, HIS3 gene, TRP1 gene, URA3 gene, ADE2 gene and LYS2 gene.

In some embodiments, the shuttle vectors further comprise a gene of interest in operable association with the archaeal promoter. In some embodiments, the gene of interest is inserted at said cloning site. The present invention is not limited to the use of any particular gene of interest. In some embodiments, the gene of interest encodes an enzyme. In some embodiments, the gene of interest has been mutated so that a screen for a specific activity of the mutated protein encoded by the gene of interest can be performed. In some embodiments, the enzyme is selected from the group consisting of a cellulase, chitinase, xylanase, pectinase, lipase and esterase.

In some embodiments, the shuttle vector is pYS3. In some embodiments, the shuttle vector is encoded by SEQ ID NO: 1.

In some embodiments, the present invention provided a host cell comprising the shuttle vector described above. The present invention is not limited to any particular host cell. In some embodiments, the host cell is selected from the group consisting of members of the genera Thermococcus and Pyrococcus.

In some embodiments, the present invention provides methods of expressing a gene of interest in an archaeon comprising: culturing an archaeon comprising the shuttle vector described above under conditions suitable for expression of said gene of interest from said promoter.

In some embodiments, the present invention provides methods of producing a protein of interest encoded by a gene of interest in an archaeon comprising: culturing an archaeon comprising the shuttle vector of claim 9 under conditions suitable for expression of said protein of interest from said gene of interest. In some embodiments, the methods further comprise purifying the protein of interest and/or assaying the activity of the protein of interest.

In some embodiments, the present invention provides methods of transforming an archaeon comprising: providing a shuttle vector as described above and introducing said shuttle vector into an archaeon.

In some embodiments, the present invention provides processes for producing an energy substrate from a biomass comprising: contacting a biomass with an archaeon transformed with a vector as described above.

In some embodiments, the present invention provides methods of screening for altered protein function comprising: mutating a nucleic acid encoding a protein of interest; transforming an archaeon with said nucleic acid; screening said archaea for expression a protein of interest with a desired property. The present invention is not limited to any particular mutation method. In some embodiments, the mutating step comprises a method selected from the group consisting of error prone PCR, chemical mutagenesis, and gene shuffling. In some embodiments, the desired property is selected form the group consisting of enhanced thermostability and enhanced action on a desired substrate. In some embodiments, the methods further comprise the step of selecting and isolating said archaea expressing a protein of interest with a desired property. In some embodiments, multiple mutations are introduced into said nucleic acid of interest. In some embodiments, greater than 100,000 transformed archaea are screened.

In some embodiments, the present invention provides methods of genetically altering an archaeon comprising: transforming said archaea with a shuttle vector comprising nucleic acid sequences that are homologous to the target gene of interest, wherein said homologous sequences flank a selectable marker. In some embodiments, the methods further comprise the step of selecting for archaea expressing the selectable marker. The present invention is not limited to any particular gene of interest. In some embodiments, the target gene of interest is selected from the group consisting of membrane bound hydrogenases and aldehyde ferredoxin oxidoreductase.

In some embodiments, the present invention provides an archaeal organism produced by the methods described above. In some embodiments, the present invention provides an archaeal organism an exogenous gene, wherein the archaeon is a Pyrococcus sp. In some embodiments, the present invention provides an archaeal organism comprising a disrupted endogenous gene, wherein the archaeon is a Pyrococcus sp.

In some embodiments, the present invention provides for the use of the foregoing vectors to transform an organism, producing a transformed organism. In some embodiments, the present invention provides for use of organisms transformed with the present invention in the methods described above, and in particular for use of the transformed organisms in industrial processes, including, but not limited to, treatment and/or fermentation of biomass, production of protein (e.g., industrial enzymes), production of fatty acids, environmental remediation, and similar processes.

### DESCRIPTION OF THE FIGURES

FIG. 1 provides the sequence for pYS3 (SEQ ID NO:1)

FIG. 2 provides the sequence for pYS4 (SEQ ID NO:2)

FIG. 3 provides a plasmid map for pYS2, pYS3 and pYS4.

FIG. 4 provides a western blot assay using antibodies against RNAP subunit D.

FIG. 5 provides an SDS-PAGE analysis of overexpressed subunit D with the His₆.

FIG. 6 provides a Southern blot of P. furiosus EcoRV-digested total DNA.

FIG. 7a provides a schematic drawing of pMUR1, a plasmid designed for the introduction of a C-terminal Strep-His-Tag into subunit rpoD. FIG. 7b provides the results of a PCR analysis of the rpoD gene locus. FIG. 7c provides the results of a Western Blot analysis of the modified subunit RpoD.

FIG. 8a provides results of Ni-NTA chromatography with cell extracts containing different NaCl concentrations. FIG. 8b provides a silver stained SDS gel of the purified RNA polymerase after Superdex 200 chromatography.

Definitions

The term “nucleotide sequence of interest” refers to any nucleotide sequence (e.g., RNA or DNA), the manipulation of which may be deemed desirable for any reason by one of ordinary skill in the art. Such nucleotide sequences include, but are not limited to, coding sequences of structural genes (e.g., reporter genes, selection marker genes, drug resistance genes, enzymes, etc.), and non-coding regulatory sequences which do not encode an mRNA or protein product (e.g., promoter sequence, termination sequence, enhancer sequence, etc.).

As used herein, the term “protein of interest” refers to a protein encoded by a nucleic acid of interest.

As used herein, the term “exogenous gene” refers to a gene that is not naturally present in a host organism or cell, or is artificially introduced into a host organism or cell.

The term “gene” refers to a nucleic acid (e.g., DNA or RNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length coding sequence or of the fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences although intervening sequences were rarely detected in archaeal genes.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

As used herein, the term “variant,” when used in reference to proteins, refers to proteins encoded by partially homologous nucleic acids so that the amino acid sequence of the proteins varies. As used herein, the term “variant” encompasses proteins encoded by homologous genes having both conservative and nonconservative amino acid substitutions that do not result in a change in protein function, as well as proteins encoded by homologous genes having amino acid substitutions that cause decreased (e.g., null mutations) protein function or increased protein function.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology” and “percent identity” when used in relation to nucleic acids refers to a degree of complementarity. There may be partial homology (i.e., partial identity) or complete homology (i.e., complete identity). A partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “selectable marker” refers to a gene that encodes an enzymatic activity that confers the ability to grow in medium lacking what would otherwise be an essential nutrient; in addition, a selectable marker may confer resistance to an antibiotic or drug upon the cell in which the selectable marker is expressed.

As used herein, the term “regulatory element” refers to a genetic element that controls some aspect of the expression of nucleic acid sequences. For example, a promoter is a regulatory element that facilitates the initiation of transcription of an operably linked coding region.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the term “biomass” refers to biological material which can be used as fuel or for industrial production. Most commonly, biomass refers to plant matter grown for use as biofuel, but it also includes plant or animal matter used for production of fibers, chemicals or heat. Biomass may also include biodegradable wastes that can be used as fuel. It is usually measured by dry weight. The term biomass is useful for plants, where some internal structures may not always be considered living tissue, such as the wood (secondary xylem) of a tree. This biomass became produced from plants that convert sunlight into plant material through photosynthesis. Sources of biomass energy lead to agricultural crop residues, energy plantations, and municipal and industrial wastes. The term “biomass,” as used herein, excludes components of traditional media used to culture microorganisms, such as purified starch, peptone, yeast extract but includes waste material obtained during industrial processes developed to produce purified starch. According to the invention, biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, corn steep liquor, grasses, wheat, wheat straw, barley, barley straw, grain residue from barley degradation during brewing of beer, hay, rice straw, switchgrass, waste paper, sugar cane bagasse, sorghum, soy, components obtained from processing of grains, trees, branches, roots, leaves, wood chips, sawdust, shrubs and bushes, soybean hulls, vegetables, fruits, flowers and animal manure. In one embodiment, biomass that is useful for the invention includes biomass that has a relatively high carbohydrate value, is relatively dense, and/or is relatively easy to collect, transport, store and/or handle.

As used herein, the term “biomass by-products” refers to biomass materials that are produced from the processing of biomass.

As used herein, the term “bioreactor” refers to an enclosed or isolated system for containment of a microorganism and a biomass material. The “bioreactor” may preferably be configured for anaerobic growth of the microorganism.

As used herein, the term “hyperthermophilic organism” means an organism which grows optimally at temperatures above 80° C.

As used herein, the terms “degrade” and “degradation” refer to the process of reducing the complexity of a substrate, such as a biomass substrate, by a biochemical process, preferably facilitated by microorganisms (i.e., biological degradation). Degradation results in the formation of simpler compounds such as methane, ethanol, hydrogen, and other relatively simple organic compounds (i.e., degradation products) from complex compounds. The term “degradation” encompasses anaerobic and aerobic processes, including fermentation processes.

### DESCRIPTION OF THE INVENTION

Pyrococcus furiosus is a model organism for analyses of molecular biology and biochemistry of archaea but so far no useful genetic tools for this species have been described. We report here a genetic transformation system for P. furiosus based on the shuttle vector system pYS2 from Pyrococcus abyssi. In the redesigned vector, the pyrE gene from Sulfolobus was replaced as selectable marker by the 3-hydroxy-3-methylglutaryl coenzyme A reductase gene (HMG-CoA) conferring resistance of transformants to the antibiotic simvastatin. Use of this modified plasmid resulted in the overexpression of the HMG-CoA reductase in P. furiosus, allowing the selection of strains by growth in the presence of simvastatin. The modified shuttle vector replicated in P. furious, but the copy number was one to two per chromosome. This system was used for overexpression of His₆ tagged subunit D of the RNA polymerase (RNAP) in Pyrococcus cells. Functional RNAP was purified from transformed cells in two steps by Ni-NTA and gel filtration chromatography. Our data provide evidence that expression of transformed genes can be controlled from a regulated gluconeogenetic promoter. Accordingly, the present invention provides genetic tools for the manipulation and expression of native and exogenous genes and their encoded proteins in archaea, in some preferred embodiments the order Thermococcales, and in some more preferred embodiments organisms of the genera Thermococcus and Pyrococcus.

### A. Shuttle Vectors

In some embodiments, the present invention provides shuttle vectors that allow propagation and/or cloning in a bacteria (e.g., E. coli) and propagation and/or expression in an archaeon (e.g., a Pyrococcus species). In some embodiments, the shuttle vector allows the propagation of cloned genes in bacteria prior to their introduction into archaea for expression.

In some embodiments, the shuttle vector comprises, in operable association, a bacterial replication origin, a bacterial selection marker gene, a gene segment that allows maintenance or propagation of the plasmid in an archaeon, an antibiotic resistance gene that confers selectability in an archaeon, and an archaeal promoter 5′ to a cloning site.

In some embodiments, the gene segment that allows maintenance or propagation of the plasmid in an archaeon is a rolling circle replication initiator protein gene that encodes a protein that enables maintenance or propagation in a selected archaeon. In some embodiments, the rolling circle replication initiator protein gene is selected from the group consisting of Rep74 and Rep75.

In some embodiments, the shuttle vector comprises an antibiotic resistance gene that confers selectability in an archaeon. In some embodiments, the antibiotic resistance gene that confers selectability in an archaeon is the 3-hydroxy-3-methylglutaryl coenzyme A reductase gene (HMG-CoA, SimR). In some embodiments, the antibiotic that is used for selection of transformed archaea is simvastatin.

In some embodiments, the shuttle vector comprises a bacterial replication origin. In some embodiments, the bacterial replication origin is an E. coli replication origin. In some embodiments, the replication origin is oriC. In some embodiments, the replication origin is obtained from the plasmid pUC19.

In some embodiments, the shuttle vector comprises a selectable marker that allows selection in the bacterial host cell. In some embodiments, the bacterial selection marker gene is different than said antibiotic resistance gene that confers selectability in an archaeon. In some embodiments, the bacterial selectable marker gene is an antibiotic resistance gene, for example AmpR, KanR, TetR, Neo, CAT, or hph, and confers resistance to the appropriate antibiotic, e.g., ampicillin, kanamycin, tetracycline, Zeocin, neomycin, chloramphenicol or hygromycin. In some embodiments, the bacterial selection marker gene is an auxotrophic marker gene such as the LEU2 gene, HIS3 gene, TRP1 gene, URA3 gene, ADE2 gene and LYS2 gene.

In some embodiments, the shuttle vector comprises a promoter that directs expression of a gene of interest in an archaeal host. Suitable promoters include, but are not limited to, glutamate dehydrogenase gene (gdh) promotor or the fructose-1, 6 bisphosphatase (fbp) promoter. In some embodiments, a cloning site is included that allows insertion of a gene of interest downstream or 3′ to the promoter. In some embodiments, the cloning site is a unique cloning site, i.e., a restriction enzyme consensus sequence that is present elsewhere in the shuttle vector. In some embodiments, the cloning site is a multiple cloning site sequence that includes restriction sites for a number of different restriction enzymes. In some embodiments, the promoter is a regulated promoter. For example, the sequence 5′ of the gene of interest may, in addition to the promoter, comprise elements that allow regulated expression of the gene of interest. Suitable elements include, but are not limited to, elements responsive to TrmB and TrmB-like transcriptional regulators for sugar transport and metabolism. Other examples of regulated promoters include, but are not limited to the archaeal heat shock promoter. See, e.g., Crystal structure of the archaeal heat shock regulator from Pyrococcus furiosus: a molecular chimera representing eukaryal and bacterial features. Liu W, Vierke G, Wenke A K, Thomm M, Ladenstein R.; J Mol Biol. 2007 Jun 1;369(2):474-88. This promoter is repressed under normal growth conditions and can be switched on by increasing the growth temperature.

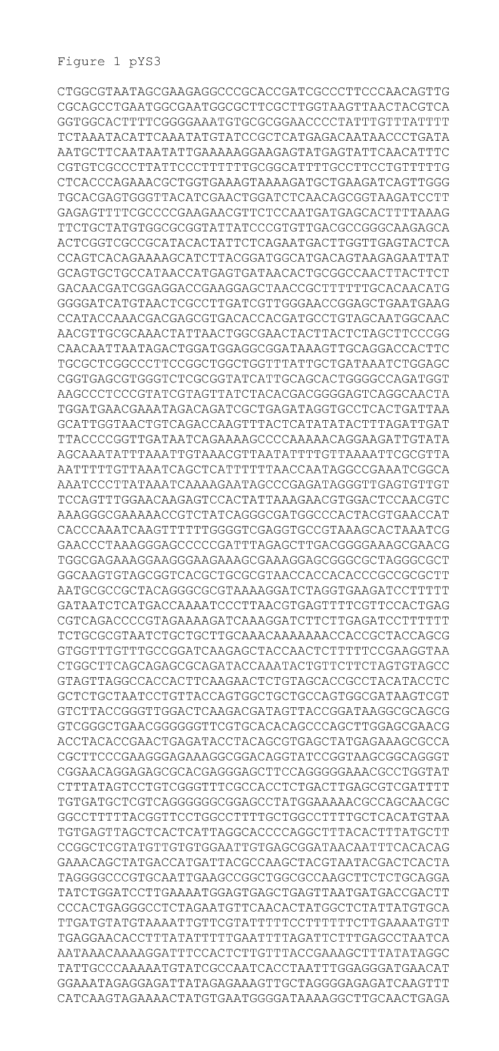

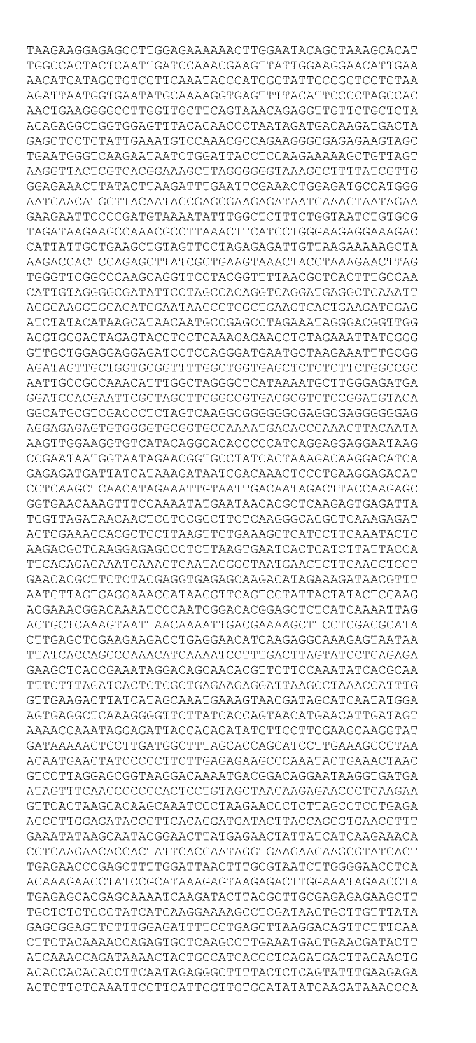

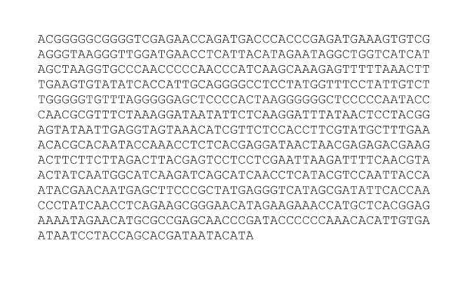

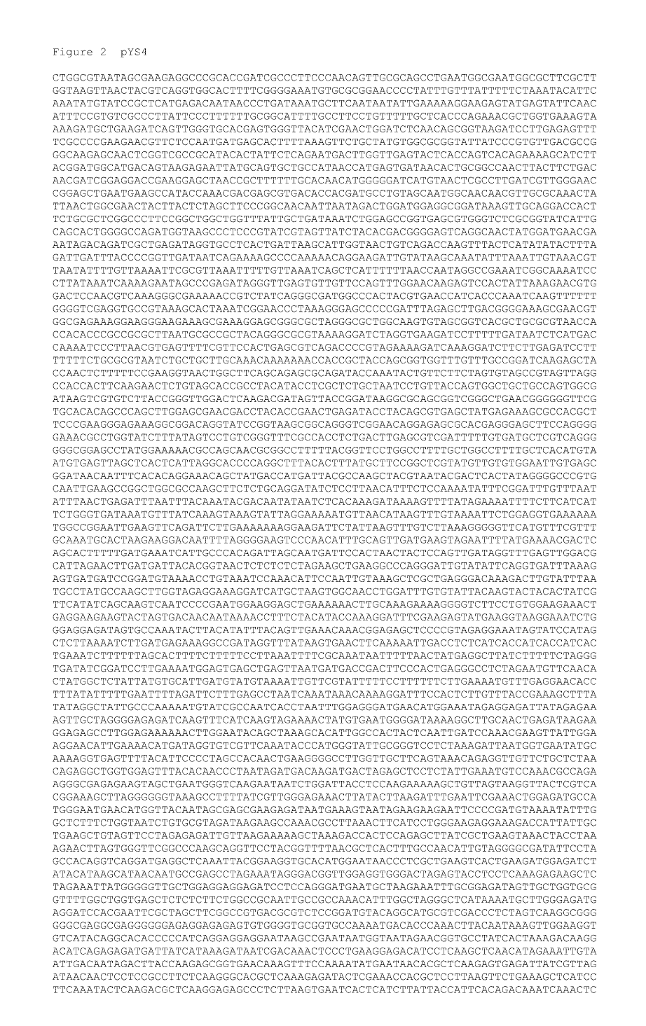

Exemplary plasmids of the present invention are described in FIGS. 1, 2 and 3 and the construction of the plasmids is described in detail in the Experimental section below. In some preferred embodiments, the present invention provides the plasmids pYS3 and pYS4. In some embodiments, the present invention provides the plasmids encoded by SEQ ID NO:1 and SEQ ID NO:2.

B. Host cells

In some embodiments, the shuttle vectors of the present invention are maintained or propagated in a bacterial host cell and an archaea host cell. In some embodiments, the bacterial host cell is Escherichia coli. In some embodiments, the archaea host cell is a thermophile or hyperthermophile. In some embodiments, the archaeal host cell belongs to the order of Thermococcales. In some embodiments, the genera Thermococcus and Pyrococcus are members of the order Thermococcales. In some embodiments, the host cell is Pyrococcus furiosus. Other examples of Pyrococcus species include, but are not limited to P. abyssi, P. endeavori, P. glycovorans, P. horikoshii, and P. woesei. Examples of Thermococcus species include, but are not limited to, T. acidaminovorans, T. aegaeus, T. aggregans, T. alcaliphilus, T. atlanticus, T. barophilus, T. barossii, T. celer, T. chitonophagus, T. coalescens, T. fumicolans, T. gammatolerans, T. gorgonarius, T. guaymasensis, T. hydrothermalis, T. kodakaraensis, T. litoralis, T. marinus, T. mexicalis, T. pacificus, T. peptonophilus, T. profundus, T. radiotolerans, T. sibiricus, and T. siculi. Accordingly, in some embodiments, the present invention provides host cells comprising a shuttle vector of the present invention. Such host cells have a variety of uses as described in more detail below. In some embodiments, the shuttle vectors of the present invention are used to transform an archaeal host cell. A number of methods of transformation are known in the art, including treatment with CaCl₂.

### C. Expression of Exogenous Genes

In some embodiments, the present invention provides methods for expressing an exogenous gene in an archaeal host cell. In some embodiments, the exogenous gene is inserted into the shuttle vector of the present invention. The present invention is not limited to the expression of any particular exogenous gene in the archaeal host cell. In some embodiments, the exogenous gene encodes a cytoplasmic protein. In some embodiments, the exogenous gene encodes a secreted protein. In some embodiments, the exogenous gene encodes an enzyme. In some embodiments, the enzyme is a thermostable enzyme. In other embodiments, the enzyme is not thermostable initially and the host cells allow for selection of thermostable variants of the enzyme.

Examples of genes of interest include, but are not limited to, cellulases. In some embodiments, the cellulase is a thermostable cellulase such as the cellulase from Pyrococcus horikoshii, See, e.g., Extremophiles. 2007 March;11(2):251-6. In some embodiments, the cellulase causes hydrolysis of carboxymethylcellulose and/or crystalline cellulose. In some embodiments, the gene of interest encodes a xylanase, pectinase, or chitinase. Xylanases catalyse hydrolysis of 1,4-D-xylosidic linkages in the hemicellulose xylan which is a major structural polysaccharide in plants and one of the most abundant polymers in nature. Xylanases are used in pulp and paper industry reducing the usage of bleaching material. A xylanase cloned in P. furiosus could enable this organism to improve growth and hydrogen yields on hemicellulose containing waste material. At present, several thermostable xylanases have been described from Thermotoga, Sulfolobus or Thermococcus which can be used for this project (Collins, T., Gerday, C., Feller, G, (2005) Xylanases, xylanase families and extremophilic xylanases. FEMS Microbiol. Rev. 29, 3-23). Two chitinases have been described for P. furiosus which allow growth of P. furiosus on colloidal chitin (Gao, J., Bauer, M. W., Shockley, K. R., Pysz, M. A., Kelly, R. M. (2003) Growth of hyperthermophilic archaeon Pyrococcus furiosus on chitin involves two family 18 chitinases. Appl. Environ. Microbiol. 69, 3119-28). Since these enzymes exist already there is no obvious need to introduce chitinases into this organism but the efficiency of these enzymes can be improved by in vitro mutagenesis and the optimized version of the chitinase encoding genes can be introduced into P. furiosus by the use of the genetic system and be used to replace the wild-type versions. In some embodiments, the gene of interest encodes a lipase or esterase. See, e.g., Expression, purification, refolding and characterization of a putative lysophospholipase from Pyrococcus furiosus: retention of structure and lipase/esterase activity in the presence of water-miscible organic solvents at high temperatures. Chandrayan S K, Dhaunta N, Guptasarma P. Protein Expr Purif. 2008 June;59(2):327-33. In some embodiments, the lipase or esterase is mutated and reintroduced into the archaeal strain for screening on selected substrates.

In some embodiments, the shuttle vector system of the present invention is used to optimize the enzymes described above as well as other enzymes of interest. In some embodiments, the present invention provides methods and systems for mutating the coding sequence of a target enzyme by in vitro evolution methods such as error prone PCR and subsequent screening for improved activity, such as screening by plate assays for improved xylanase or chitinase activities directly at the physiological growth temperature of Pyrococcus. In some embodiments, the assays are high throughput assays.

In some embodiments, the shuttle vector system of the present invention can be used for gene disruption of a target gene or gene replacement strategies. The data herein provide evidence that this system allows the construction of knockout mutants in P. furiosus by double cross-over homologous recombination. The gene PF0496 coding for a transcriptional regulator of the TrmB family (Lee, S. J., Surma, M., Hausner, W., Thomm, M., Boos, W. (2008) The role of TrmB and TrmB-like transcriptional regulators for sugar transport and metabolism in the hyperthermophilic archaeon Pyrococcus furiosus. Arch. Microbiol. 190, 247-56) was deleted by using the over expression cassette of the hmg-CoA reductase gene of Thermococcus kodakaraensis KOD1 as a selectable marker. For the transformation procedure, the hmg-CoA reductase gene was combined with about 1000 by of the corresponding upstream and downstream DNA sequences to define the cross-over positions within the genome. A Southern blot analysis of the transformant confirmed the replacement of PF0496 by the selectable marker. Further experiments demonstrated that this system allows gene disruption as well as gene modification of different genes in the chromosome of P. furiosus. Therefore, it is also possible to use this system to modify the metabolism of Pyrococcus to improve the biotechnological potential for biomass conversion.

In some embodiments, the present invention provides methods of making and using genetically engineered archaea, such as P. furiosus, to produce ethanol. When Pyrococcus furiosus is grown on polymers like starch and polypeptides, glucose is degraded to pyruvate via a modified Embden Meyerhof pathway (Schönheit, P. (2008) Glycolysis in hyperthermophiles in: Robb, F., Antranikian, G., Grogan, D., and Driessen, A. (eds.) Thermophiles: Biology and Technology at high temperatures. Pp.99-112, CRC Press, Boca Raton, London, New York) to acetate, hydrogen and CO₂ as major products and ethanol is formed in very low amounts. The existence of a genetic knockout system for P. furiosus facillitates alteration of the metabolism from acetate and hydrogen production to ethanol formation.

Pyruvate is converted to Acetyl-CoA and CO₂ by the activity of the enzyme pyruvate ferredoxin oxidoreductase (POR). This enzyme is also able to produce acetaldehyde in a CoA dependent reaction (Ma, K., Hutchins, A., Sung, S. J., Adams, M. W. (1997) Pyruvate ferredoxin oxidoreductase from the hyperthermophilic archaeon, Pyrococcus furiosus, functions as a CoA-dependent pyruvate decarboxylase. Proc. Natl. Acad. Sci. USA. 94, 9608-13.) which can be converted to ethanol by the P. furiosus alcohol dehydrogenase (ADH):
CH₃-CHO+NADPH [symbol] CH₃-CH₂OH+NADP⁺
   (van der Oost, J., Voorhorst, W. G., Kengen, S. W., Geerling, A. C., Wittenhorst, V., Gueguen, Y., de Vos, W. M. Genetic and biochemical characterization of a short-chain alcohol dehydrogenase from the hyperthermophilic archaeon Pyrococcus furiosus. Eur. J. Biochem. 268, 3062-8, 2001). The formation of up to 1 mM ethanol in P. furiosus cultures grown on potato pulp and corn silage as substrate has been observed. This finding indicates that the POR activity synthesizing acetaldehyde in vitro and the presence of ADH reported in P. furiosus cultures lead actually to formation of ethanol in P. furiosus cells. A second aldehyde-utilizing enzyme is aldehyde ferredoxin oxidoreductase (AOR) catalyzing the reaction:
Acetaldehyde+Fdₒₓ [symbol] Acetate+Fd_{red}
Acetyl CoA is converted to acetate by the acetyl-CoA-synthase:
Acetyl-CoA+ADP+P [symbol] Acetate+ATP+CoA

P. furiosus has an anaerobic respiratory system consisting of a single membrane bound hydrogenase. This enzyme reduces protons to hydrogen and this reaction generates a protone motive force used for ATP synthesis (Sapra, R., Bagramyan, K., Adams, M. W. (2003) A simple energy-conserving system: proton reduction coupled to proton translocation. Proc. Natl. Acad. Sci. USA. 100, 7545-50). Reduced cytoplasmatic ferrodoxin is used as electron donor for this reaction and this reaction seems to be coupled with oxidation of gyceraldehdye-3-phosphate to 3-phosphoglycerate by the glyceraldehyde:Ferredoxin oxidoreductase (Mukund, S., Adams, M. W. (1995) Glyceraldehyde-3-phosphate ferredoxin oxidoreductase, a novel tungsten-containing enzyme with a potential glycolytic role in the hyperthermophilic archaeon Pyrococcus furiosus. J. Biol. Chem. 270, 8389-92).

In some embodiments, the present invention utilizes a genetic knockout system to reduce the activity of enzymes producing acetic acid and eliminate hydrogen producing enzymes (hydrogenases) to shift the metabolism of an archaea such as Pyrococcus from acetate and hydrogen formation to the production of large amounts of ethanol. In some embodiments, the AOR and/or the membrane bound hydrogenase are knocked out. In some embodiments, when the AOR is disrupted by genetic manipulation it is likely that the reducing equivalents in accumulating Fd_{red} will be used to reduce NADP⁺ to NADPH and stimulate ethanol production. The same is true when hydrogenases producing H₂ and Fdₒₓ are deleted. It is contemplated that in AOR and hydrogenase mutants (and combinations of double, triple and multiple deletion mutants of this kind) a significant fraction of glucose is converted into ethanol at the expense of acetate and hydrogen. Since the major acetate producing enzyme, the acetyl-CoA-synthase (ACD), couples acetate formation with the formation of ATP, it is unlikely that metabolism can be directed completely from generation of acetate to ethanol unless different energy yielding reactions exist as bypass reaction in P. furiosus cells or are introduced by genetic engineering. At the high growth temperature of P. furiosus of ˜90-100° C. the produced ethanol evaporates spontaneously from the liquid phase and can be easily recovered by cooling of the distillate.

### D. Uses of Transformed Archaeal Host Cells

Transformed archaeal host cells have a variety of uses. In some embodiments, the transformed host cells are used for the production of a protein of interest. In some embodiments, the protein of interest is encoded by a gene of interest operably associated with an archaeal promoter as described above. In some embodiments, the protein if interest is secreted, while in other embodiments, the protein of interest is intracellular. In some embodiments, the protein if interest is purified or separated from the host cells. In the case of intracellular proteins, the host cells are preferably disrupted during the separation or purification procedure.

In some embodiments, the genetic systems of the present invention allow for the selection of proteins with thermostable characteristics. In some embodiments, a gene encoding a protein of interest that lacks substantial thermostable characteristics is introduced into a hyperthermophilic host cell and expressed. In some embodiments, as described above, the host cells are mutagenized artificially or allowed to mutagenize and then cells expressing variants of the protein of interest that have acquired thermostable characteristics are isolated. In some embodiments, the host cells are exposed to a selective pressure that favors selection of thermostable variants of the protein of interest. Following selection, the variants can be cloned, sequenced, and analyzed.

In some embodiments, the transformed host cells are used to degrade a biomass. Suitable methods are described in co-pending applications 11/879,710 and 12/566,282, both of which are incorporated herein by reference in their entirety.

The present invention contemplates the degradation of biomass with transformed hyperthermophilic organisms. The present invention is not limited to the use of any particular biomass or organic matter. Suitable biomass and organic matter includes, but is not limited to, sewage, agricultural waste products, brewery grain by-products, food waste, organic industry waste, forestry waste, crops, grass, seaweed, plankton, algae, fish, fish waste, corn potato waste, sugar cane waste, sugar beet waste, straw, paper waste, chicken manure, cow manure, hog manure, switchgrass and combinations thereof. In some embodiments, the biomass is harvested particularly for use in hyperthermophilic degradation processes, while in other embodiments waste or by-products materials from a pre-existing industry are utilized.

In some preferred embodiments, the biomass is lignocellulosic. In some embodiments, the biomass is pretreated with cellulases or other enzymes to digest the cellulose. In some embodiments, the biomass is pretreated by heating in the presence of a mineral acid or base catalyst to completely or partially hydrolyze hemicellulose, de-crystallize cellulose, and remove lignin. This allows cellulose enzymes to access the cellulose. In some embodiments, the transformed hyperthermophillic organism is transformed with an enzyme that enhances degradation of the biomass, for example, a cellulase or lignase.

In still other preferred embodiments, the biomass is supplemented with minerals, energy sources or other organic substances. Examples of minerals include, but are not limited, to those found in seawater such as NaCl, MgSO₄x7 H₂O, MgCl₂x6 H₂O, CaCl₂x2 H₂O, KCl, NaBr, H₃BO₃, SrCl₂x6 H₂O and KI and other minerals such as MnSO₄xH₂O, Fe SO₄x7 H₂O, Co SO₄x7 H₂O, Zn SO₄x7 H₂O, Cu SO₄x5 H₂O, KA1(SO₄)₂x12H₂O, Na₂MoO₄x2 H₂O, (NH₄)₂Ni(SO₄)₂x6 H₂O, Na₂WO₄x2 H₂O and Na₂SeO₄.

Examples of energy sources and other substrates include, but are not limited to, purified sucrose, fructose, glucose, starch, peptone, yeast extract, amino acids, nucleotides, nucleosides, and other components commonly included in cell culture media.

In other embodiments, the biomass that is utilized has been previously fermented by another process. Surprisingly, it has been found that hyperthermophilic organisms are capable of growing on biomass that has been previously fermented by methanogenic microorganisms.

In some embodiments, biomass that contains or is suspected of containing human pathogens is treated by the hyperthermophilic process to destroy the pathogenic organisms. In some preferred embodiments, the biomass is heated to about 80° C. to 120° C., preferably to about 100° C. to 120° C., for a time period sufficient to render pathogens harmless. In this manner, waste such a human sewage may be treated so that it can be further processed to provide a safe fertilizer, soil amendment of fill material in addition to other uses.

In some preferred embodiments, the biomass is an algae, most preferably a marine algae (seaweed). In some embodiments, marine algae is added to another biomass material to stimulate hydrogen and/or acetate production. In some embodiments, the biomass substrate comprises a first biomass material that is not marine algae and marine algae in a concentration of about 0.01% to about 50%, weight/weight (w/w), preferably 0.1% to about 50% w/w, about 0.1% to about 20% w/w, about 0.1% to about 10% w/w, about 0.1% to about 5% w/w, or about preferably 1.0% to about 50% w/w, about 1.0% to about 20% w/w, about 1.0% to about 10% w/w, or about 1.0% to about 5% w/w. The present invention contemplates the use of a wide variety of seaweeds, including, but not limited to, marine prokaryotes such as cyanobacteria (blue-green algae), green algae (division Chlorophyta), brown algae (Phaeophyceae, division Phaeophyta), and red algae (division Rhodophyta, e.g. Palmaria palmata). In some embodiments, the brown algae is a kelp, for example, a member of genus Laminaria (Laminaria sp.), such as Laminaria hyperborea, Laminaria digitata, Laminaria abyssalis, Laminaria agardhii, Laminaria angustata, Laminaria appressirhiza, Laminaria brasiliensis, Laminaria brongardiana, Laminaria bulbosa, Laminaria bullata, Laminaria complanata, Laminaria dentigera, Laminaria diabolica, Laminaria ephemera, Laminaria farlowii, Laminaria hyperborea, Laminaria inclinatorhiza, Laminaria multiplicata, Laminaria ochroleuca, Laminaria pallid, Laminaria platymeris, Laminaria rodriguezii, Laminaria ruprechtii, Laminaria sachalinensis, Laminaria setchellii, Laminaria sinclairii, Laminaria solidugula and Laminaria yezoensis or a member of the genus Saccharina (Saccharina sp.), such as Saccharina angustata, Saccharina bongardiana, Saccharina cichorioides, Saccharina coriacea, Saccharina crassifolia, Saccharina dentigera, Saccharina groenlandica, Saccharina gurjanovae, Saccharina gyrate, Saccharina japonica, Saccharina kurilensis, Saccharina latissima, Saccharina longicruris, Saccharina longipedales, Saccharina longissima, Saccharina ochotensis, Saccharina religiosa, Saccharina sculpera, Saccharina sessilis, and Saccharina yendoana. In some embodiments, the brown algae if from one of the following following genera: Fucus, Sargassum, and Ectocarpus.

In preferred embodiments of the present invention, one or more populations of hyperthermophilic organisms are utilized to degrade biomass. In some embodiments, the biomass is transferred to a vessel such as a bioreactor and inoculated with one or more strains of hyperthermophilic organisms. In some embodiments, the environment of the vessel is maintained at a temperature, pressure, redox potential, and pH sufficient to allow the strain(s) to metabolize the feedstock. In some preferred embodiments, the environment has no added sulfur or inorganic sulfide salts or is treated to remove or neutralize such compounds. In other, embodiments, reducing agents, including sulfur containing compounds, are added to the initial culture so that the redox potential of the culture is lowered. In some preferred embodiments, the environment is maintained at a temperature above 45° C. In still further embodiments, the environment is maintained at between 55 and 90° C. In still further embodiments, the culture is maintained at from about 80° C. to about 110° C. depending on the hyperthermophilic organism utilized. In some preferred embodiments, sugars, starches, xylans, celluloses, oils, petroleums, bitumens, amino acids, long-chain fatty acids, proteins, or combinations thereof, are added to the biomass. In some embodiments, water is added to the biomass to form an at least a partially aqueous medium. In some embodiments, the aqueous medium has a dissolved oxygen gas concentration of between about 0.2 mg/liter and 2.8 mg/liter. In some embodiments, the environment is maintained at a pH of between approximately 4 and 10. In some embodiments, the environment is preconditioned with an inert gas selected from a group consisting of nitrogen, carbon dioxide, helium, neon, argon, krypton, xenon, and combinations thereof. While in other embodiments, oxygen is added to the environment to support aerobic degradation.

In other embodiments, the culture is maintained under anaerobic conditions. In some embodiments, the redox potential of the culture is maintained at from about −125 mV to −850 my, and preferably below about −500 mV. Surprisingly, in some embodiments, the redox potential is maintained at a level so that when a biomass substrate containing oxygen is added to an anaerobic culture, any oxygen in the biomass is reduced thus removing the oxygen from the culture so that anaerobic conditions are maintained.

In some embodiments, where lignocellulosic materials are utilized, the cellulose is pre-treated as described above. The pre-treated cellulose is enzymatically hydrolyzed either prior to degradation in sequential saccharification and degradation or by adding the cellulose and hyperthermophile inoculum together for simultaneous saccharification and degradation.

It is contemplated that degradation of the biomass with the transformed hyperthermophilic organisms will both directly produce energy in the form of heat (i.e., the culture is exothermic or heat-generating) as well as produce products that can be used in subsequent processes, including the production of energy. In some embodiments, hydrogen, methane, and ethanol are produced by the degradation and utilized for energy production. In preferred embodiments, these products are removed from the vessel. It is contemplated that removal of these materials in the gas phase will be facilitated by the high temperature in the culture vessel. These products may be converted into energy by standard processes including combustion and/or formation of steam to drive steam turbines or generators. In some embodiments, the hydrogen is utilized in fuel cells. In some embodiments, proteins, acids and glycerol are formed which can be purified for other uses or, for example, used as animal feeds.

In some embodiments, the culture is maintained so as to maximize hydrogen production. In some embodiments, the culture is maintained under anaerobic conditions and the population of microorganisms is maintained in the stationary phase. Stationary phase conditions represent a growth state in which, after the logarithmic growth phase, the rate of cell division and the one of cell death are in equilibrium, thus a constant concentration of microorganisms is maintained in the vessel.

In some embodiments, the degradation products are removed from the vessel. It is contemplated that the high temperatures at which the degradation can be conducted facilitate removal of valuable degradation products from the vessel in the gas phase. In some embodiments, methane, hydrogen and/or ethanol are removed from the vessel. In some embodiments, these materials are moved from the vessel via a system of pipes so that the product can be used to generate power or electricity. For example, in some embodiments, methane or ethanol are used in a combustion unit to generate power or electricity. In some embodiments, steam power is generated via a steam turbine or generator. In some embodiments, the products are packages for use. For example, the ethanol, methane or hydrogen can be packaged in tanks or tankers and transported to a site remote from the fermenting vessel. In other embodiments, the products are fed into a pipeline system.

In still other embodiments, heat generated in the vessel is utilized. In some embodiments, the heat generated is utilized in radiant system where a liquid is heated and then circulated via pipes or tubes in an area requiring heating. In some embodiments, the heat is utilized in a heat pump system. In still other embodiments, the heat is utilized to produce electricity via a thermocouple. In some embodiments, the electricity produced is used to generate hydrogen via an electrolysis reaction.

In other preferred embodiments, the excess heat generated by the fermentation process is used to generate electricity in an Organic Rankine Cycle (ORC). A Rankine cycle is a thermodynamic cycle which converts heat into work. The heat is supplied externally to a closed loop, which usually uses water as the working fluid to drive a turbine coupled to the system. Conventional Rankine cycle processes generate about 80% of all electric power used in America and throughout the world, including virtually all solar thermal, biomass, coal and nuclear power plants. The organic Rankine cycle (ORC) uses an organic fluid such as pentane or butane in place of water and steam. This allows use of lower-temperature heat sources, which typically operate at around 70-90° C. Heat from the bioreactor, which runs at approximately 80° C., is used to heat an organic solvent such as perfluor pentane in a closed loop. The heated solvent expands through a turbine and generates electricity via the generator. The solvent cools and is passed though a condenser.

In other preferred embodiments, the present invention provides a process in which biomass is treated in two or more stages with transformed hyperthermophilic organisms. In some embodiments, the process comprise a first stage where a first hyperthermophilic organism is used to treat a biomass substrate, and a second stage where a second hyperthermophilic organism is used to treat the material produced from the first stage. Additional hyperthermophilic degradation stages can be included. In some embodiments, the first stage utilizes Pyroccoccus furiosus, while the second stage utilizes Thermotoga maritima. In some preferred embodiments, the material produced from the second stage, including acetate, is further utilized as a substrate for methane production as described in more detail below.

In some embodiments, H₂ and/or CO₂ produced during hyperthermophilic degradation of a biomass are combined with methane from a biogas facility to provide a combustible gas. In some embodiments, H₂ and/or CO₂ producing during hyperthermophilic degradation of a biomass are added to a biogas reactor to increase production of methane.

The present invention also provides systems, compositions and processes for degrading biomass under improved conditions. In some embodiments, a hyperthermophile strain derived from a marine hyperthermophile is utilized and the biomass is provided in a liquid medium that comprises less than about 0.2% NaCl. In some embodiments, the NaCl concentration ranges from about 0.05% to about 0.2%, preferably about 0.1% to about 0.2%. In some embodiments, the preferred strain is MH-2 (Accession No. DSM 22926). In these embodiments, the biomass is suspended in a liquid medium so that it can be pumped into a bioreactor system. It is contemplated that the lower salt concentration allows use of the residue left after degradation for a wider variety of uses and also results in less corrosion of equipment. Furthermore, the lower salt concentration allows for direct introduction of the degraded biomass containing acetate, or liquid medium containing acetate that is derived from the hyperthermophilic degradation, into a biogas reactor.

In further embodiments, the processes and microorganisms described herein facilitate degradation of biomass using concentrations of hyperthermophilic organisms that have not been previously described. In some embodiments, the concentration of the hyperthermophilic organism in the bioreactor is greater than about 10⁹ cells/ml. In some embodiments, the cell concentration ranges from about 10⁹ cells/ml to about 10¹¹ cells/ml, preferably from about 10⁹ cells/ml to about 10¹⁰ cells/ml.

In still further embodiments, the present invention provides processes that substantially decrease the hydraulic retention time of a given amount of biomass in a reactor. Hydraulic retention time is a measure of the average length of time that a soluble compound, in this case biomass suspended or mixed in a liquid medium, remains in a constructed reactor and is presented in hours or days. In some embodiments, the hydraulic retention time of biomass material input into a bioreactor in a process of the present invention is less than about 10 hours, preferably less than about 5 hours, more preferably less than about 4 hours, and most preferably less than about 3 or 2 hours. In some embodiments, the hydraulic retention time in a hyperthermophilic degradation process of the present invention is from about 1 to about 10 hours, preferably from about 1 to 5 hours, and most preferably from about 2 to 4 hours.

In some embodiments, the transformed hyperthermophilic organisms are used to treat or process a hydrocarbon composition. Examples of hydrocarbon compositions include, but are not limited to crude oil, produced water from oil wells, produced water from coal bed methane, oil sand, oil shale, oil waste water, coal waste water, and the like.

Produced water is water trapped in underground formations that is brought to the surface along with oil or gas. It is by far the largest volume byproduct or waste stream associated with oil and gas production. Management of produced water presents challenges and costs to operators. According to the American Petroleum Institute (API), about 18 billion barrels (bbl) of produced water was generated by U.S. onshore operations in 1995 (API 2000). Additional large volumes of produced water are generated at U.S. offshore wells and at thousands of wells in other countries. Khatib and Verbeek (2003) estimate that for 1999, an average of 210 million bbl of water was produced each day worldwide. This volume represents about 77 billion bbl of produced water for the entire year.

In subsurface formations, naturally occurring rocks are generally permeated with fluids such as water, oil, or gas (or some combination of these fluids). It is believed that the rock in most oil-bearing formations was completely saturated with water prior to the invasion and trapping of petroleum (Amyx et al. 1960). The less dense hydrocarbons migrated to trap locations, displacing some of the water from the formation in becoming hydrocarbon reservoirs. Thus, reservoir rocks normally contain both petroleum hydrocarbons (liquid and gas) and water. Sources of this water may include flow from above or below the hydrocarbon zone, flow from within the hydrocarbon zone, or flow from injected fluids and additives resulting from production activities. This water is frequently referred to as “connate water” or “formation water” and becomes produced water when the reservoir is produced and these fluids are brought to the surface. Produced water is any water that is present in a reservoir with the hydrocarbon resource and is produced to the surface with the crude oil or natural gas.

When hydrocarbons are produced, they are brought to the surface as a produced fluid mixture. The composition of this produced fluid is dependent on whether crude oil or natural gas is being produced and generally includes a mixture of either liquid or gaseous hydrocarbons, produced water, dissolved or suspended solids, produced solids such as sand or silt, and injected fluids and additives that may have been placed in the formation as a result of exploration and production activities. Production of coal bed methane (CBM) involves removal of formation water so that the natural gas in the coal seams can migrate to the collection wells. This formation water is also referred to as produced water. It shares some of the same properties as produced water from oil or conventional gas production, but may be quite different in composition.

Accordingly, in some embodiments, the present invention further provides methods comprising: a) providing a hydrocarbon composition and a population of at least one genus of a transformed hyperthermophilic organism; and b) treating the hydrocarbon composition in the presence of the population of at least one genus of a transformed hyperthermophilic organism under conditions such that degradation products are produced. In some preferred embodiments, the anaerobic transformed hyperthermophilic organisms are selected from the group consisting of the archaeal genera Pyrococcus, Thermococcus, Palaeococcus, Acidianus, Pyrobaculum, Pyrolobus, Pyrodictium, Methanopyrus, Methanothermus, Methanobacterium, hyperthermophilic Methanococci like Methanocaldococcus jannaschii, Archaeoglobus, and of the bacterial genera Thermosipho, Thermotoga, Fervidobacterium, Thermodesulfobacterium and combinations thereof. In some preferred embodiments, the hydrocarbon composition is selected from the group consisting of produced water from oil wells, oil sand, oil shale, oil waste water, coal waste water, and the like, and combinations thereof. In some embodiments, the hydrocarbon composition is supplemented with a biomass component such as those described in detail above and/or a cell culture media component selected from the group consisting of a mineral source, vitamins, amino acids, an energy source, and a microorganism extract. In some further preferred embodiments, the degradation products are selected from the group consisting of hydrogen, methane and ethanol. In some embodiments, the methods further comprise the step of converting the degradation products into energy. In some embodiments, the methods further comprise the step of using the hydrogen in a fuel cell. In some embodiments, the methods further comprise the step of using the methane or ethanol in a combustion unit.

In other embodiments, the present invention provides methods of treating oil wells or oil bearing formations with transformed hyperthermophilic organisms. In these embodiments, a composition comprising active or dormant transformed hyperthermophilic organisms is injected into an oil well or into an oil bearing formation via an oil well, injection well or bore hole. The producer bore hole in an oil well is generally lined in the hydrocarbon bearing stratum with“gravel packs”, sand containing filter elements, which serve to trap formation fragments and it has been proposed to include in such gravel packs ceramic particles coated with or impregnated with well treatment chemicals such as scale inhibitors (see EP-A-656459 and WO 96/27070) or bacteria (see WO 99/36667). Likewise treatment of the formation surrounding the producer well bore hole with well treatment chemicals before hydrocarbon production begins has also been proposed, e. g. in GB-A-2290096 and WO 99/54592.

In some preferred embodiments, the transformed anaerobic hyperthermophilic organisms are selected from the group consisting of the genera consisting of the archaeal genera Pyrococcus, Thermococcus, Palaeococcus, Acidianus, Pyrobaculum, Pyrolobus, Pyrodictium, Methanopyrus, Methanothermus, Methanobacterium, hyperthermophilic Methanococci like Methanocaldococcus jannaschii, Archaeoglobus, and of the bacterial genera Thermosipho, Thermotoga, Fervidobacterium, Thermodesulfobacterium and combinations thereof. In some embodiments, the composition comprising transformed hyperthermophilic organisms comprises a medium that facilitates growth of the transformed hyperthermophilic organism, including energy substrates and other culture components such as mineral, salts, vitamins, amino acids, and/or microorganism extracts such as yeast extracts. In some embodiments, the compositions comprise a biomass substrate such as those described in detail above. In some embodiments, the composition comprising transformed hyperthermophilic organisms is packaged in a vehicle that allows delivery via an oil well and designed to release its contents at a predetermined location within the well, such as at the site of an oil bearing formation. In some embodiments, the compositions further comprise a matrix for delivery of the transformed hyperthermophilic organisms. Various polymeric, oligomeric, inorganic and other particulate carriers for well treatment chemicals are also known, e. g. ion exchange resin particles (see U.S. Pat. No. 4,787,455), acrylamide polymer particles (see EP-A-193369), gelatin capsules (see U.S. Pat. No. 3,676,363), oligomeric matrices and capsules (see U.S. Pat. No. 4,986,353 and U.S. Pat. No. 4,986,354), ceramic particles (see WO 99/54592, WO 96/27070 and EP-A-656459), and particles of the well treatment chemical itself (see WO 97/45625). These particles may be adapted for delivery of hyperthermophilic organisms.

In the method of the invention the compositions comprising transformed hyperthermophilic organisms may be placed down hole before and/or after hydrocarbon production (i. e. extraction of oil or gas from the well) has begun. In some embodiments, the bacteria are placed down hole before production has begun, especially in the completion phase of well construction.

The compositions comprising transformed hyperthermophilic organisms may be placed within the bore hole (e. g. in the hydrocarbon bearing strata or in ratholes) or within the surrounding formation (e. g. in fissures or within the rock itself). In the former case, the compositions comprising transformed hyperthermophilic organisms are conveniently impregnated into particles contained within a tubular filter, e.g., a gravel pack or a filter structure as disclosed in EP-A-656459 or WO 96/27070 ; in the latter case, the compositions comprising transformed hyperthermophilic organisms (optionally impregnated into particles) are preferably positioned by squeezing a liquid composition comprising transformed hyperthermophilic organisms down the bore hole. Preferably, before production begins the compositions comprising hyperthermophilic organisms are placed both within the bore in a filter and within the surrounding formation. The transformed hyperthermophilic organisms are alternatively inoculated into the particles.

Where the transformed hyperthermophilic organisms (typically impregnated into particles) are placed within the surrounding formation, the pressure used should be sufficient to cause the bacteria to penetrate at least lm, more preferably at least 1.5 m, still more preferably at least 2 m, into the formation. If desired, the transformed hyperthermophilic organisms may be applied in conjunction with porous particles to achieve a penetration of about 2 m or more into the formation.

Compositions comprising such small, porous particles and bacteria according to the invention, which may be co-blended with nutrients, form a further aspect of the invention.

Particles soaked or loaded (also referred to herein as impregnated) with transformed hyperthermophilic organisms according to the invention advantageously have mode particle sizes (e.g., as measured with a Coulter particle size analyzer) of 1 Am to 5 mm, more preferably 10 Am to 1000 ym, especially 250 to 800/mi. For placement within the formation, the mode particle size is preferably 1 to 50 ym, especially 1 to 20 Am e. g. 1-5 Am. For any particular formation, formation permeability (which correlates to the pore throat sizes in the formation) may readily be determined using rock samples taken during drilling and the optimum impregnated particle size may thus be determined. Since the particles produced as described in EP-B-3905, U.S. Pat. No. 4,530,956 and WO 99/19375 have a very low dispersity (i. e. size variation), a highly uniform deposition and deep penetration into the formation can be achieved. For this reason, the particles preferably have a coefficient of variation (CV) of less than 10%, more preferably less than 5%, still more preferably less than 2 W.

CV is determined in percentage as CV=100×standard deviation mean where mean is the mean particle diameter and standard deviation is the standard deviation in particle size. CV is preferably calculated on the main mode, i. e. by fitting a monomodal distribution curve to the detected particle size distribution. Thus some particles below or above mode size may be discounted in the calculation which may for example be based on about 90% of total particle number (of detectable particles that is). Such a determination of CV is performable on a Coulter LS 130 particle size analyzer.

For placement in filters, the impregnated particles preferably have mode particle sizes of 50 to 5000 ym, more especially 50 to 1000 Um, still more preferably 100 to 500 Am. In such filters, the impregnated particles preferably constitute 1 to 99% wt, more preferably 2 to 30% wt, still more preferably 5 to 20% wt of the particulate filter matrix, the remaining matrix comprising particulate oil-and water-insoluble inorganic material, preferably an inorganic oxide such as silica, alumina or alumina-silica. Particularly preferably, the inorganic oxide has a mode particle size which is similar to that of the impregnated polymer particles, e. g. within 20%, more preferably within 10%.

As with the in-formation placement, the impregnated particles preferably have low dispersity, e. g. a CV of less than 10%, more preferably less than 5%, still more preferably less than 2 W. The low dispersity serves to hinder clogging of the filters.

The pores of the particles will be large enough to allow the microorganisms to penetrate without difficulties e. g. a pore radius of up to 2-4 ym. The impregnated particles are preferably particles having a pore volume of at least 50%, more preferably at least 70%, e. g up to at least 85%.

The bacterially impregnated polymer particles used according to the invention, e. g. MPP or other step-grown polymer particles are preferably vinyl homo-and copolymers more preferably styrenic homo-and copolymers. Examples of appropriate monomers include vinyl aliphatic monomers such as esters of acrylic and methacrylic acids, acrylonitrile, and vinyl aromatic monomers such as styrene and substituted styrenes.

Preferred polymers are styrenic polymers, optionally and preferably cross-linked, e. g. with divinyl benzene, and particles of such polymers are commercially available in a range of sizes and pore volumes from Dyno Specialty Polymers AS of Lillestrm, Norway. If desired, the particles may be functionalized, e. g. to provide surface acidic or basic groups (e. g. carboxyl or amino functions), for example to scavenge metal atoms from water reaching the particles so as to reduce scale formation, to promote particle adhesion to formation surfaces, to promote or hinder particle aggregation, etc. Again functionalized particles are available from Dyno Specialty Polymers AS.

Preferably the polymer matrix of the impregnated particles has a softening point above the temperatures encountered down hole, e. g. one above 70° C., more preferably above 100° C., still more preferably above 150° C.

Generally where the particles are impregnated with transformed hyperthermophilic organisms, they will also be impregnated with nutrients for the bacteria, e. g. sucrose, so that bacterial growth is promoted once the particles encounter water.

Examples of typical well treatment chemicals, precursors and generators are mentioned in the patent publications mentioned herein, the contents of all of which are hereby incorporated by reference.

Thus for example typical scale inhibitors include inorganic and organic phosphonates (e. g. sodium aminotrismethylenephosphonate), polyaminocarboxylic acids or copolymers thereof, polyacrylamines, polycarboxylic acids, polysulphonic acids, phosphate esters, inorganic phosphates, polyacrylic acids, inulins (e. g. sodium carboxymethyl inulin), phytic acid and derivatives (especially carboxylic derivatives) thereof, polyaspartates, etc. The use of environmentally friendly scale inhibitors, e. g. inulins, phytic acid and derivatives thereof and polyaspartates, is especially preferred.

Where the scale inhibitor is a polymer it may of course contain residues of one or more different comonomers, e. g. a copolymer of aspartic acid and proline.

Other beneficial microbial products include enzymes which are themselves able to synthesize well treatment chemicals such as scale inhibitors. It may be necessary to transform the bacteria with a plurality of genes coding for different enzymes which are involved in a synthetic pathway for a described well treatment chemical. Thus the well treatment chemical may be directly produced by the Archaea, i. e. an expression product, or indirectly produced as a result of metabolism or catabolism within the Archaea. Thus the well treatment chemical may be proteinaceous e. g. a polypeptide or glycoprotein but it need not be and could be a polysaccharide or a lipid.

Thus in a further aspect, the present invention also provides a method for the treatment of a hydrocarbon well which method comprises administering down an injection well transformed hyperthermophilic archaea.

Where the transformed hyperthermophilic organisms are placed within the formation, they are preferably applied as a dispersion in a liquid carrier. For pre-and post-completion application, the liquid carrier preferably comprises a non-aqueous organic liquid, e. g. a hydrocarbon or hydrocarbon mixture, typically a C3 to C15 hydrocarbon, or oil, e. g. crude oil. For curative treatment, i. e. after production has continued for some time, the liquid carrier may be aqueous or non-aqueous. Impregnation of the bacteria and if desired nutrients and/or other well treatment chemicals into porous carrier particles may be effected by any conventional manner, e. g. by contacting the particles with an aqueous or non-aqueous dispersion of the bacteria or other chemicals followed if necessary by solvent removal, e. g. by draining, drying or under vacuum.

However it is especially preferred to impregnate particles with the bacteria by slurry mixing, i. e. by adding a quantity of dispersion which is close to the pore volume of the particles, e. g. 0.8 to 1.2 times pore volume more preferably 0.9 to 1.1 times pore volume. Still more preferred is to impregnate the particles by a soaking procedure using a vacuum. The process may conveniently be performed in a rotavapor at 0-15 mbar at room temperature and continued at 50° C. until most of the water-phase has been removed. It is desirable to introduce bacteria into the pore system not only onto the surface. If desired particle loading may be increased by carrying out more than one impregnation step.

Various methods can be envisaged to sustain the microorganism population in situ. The microorganism can be immobilized in the porous matrix with nutrition packages or co-injected with nutrients into small porous particles which can then be injected deep (e. g. 2-10 m) into the formation. High concentration inoculates of the transformed hyperthermophilic bacteria can be introduced into the porous particles. Advantageously, some of the bacterial species which may be introduced are capable of producing viable spores in the well environment.

The invention also includes a bioreactor for cultivating transformed hyperthermophilic organisms. The well treatment substrates and/or transformed hyperthermophilic organisms are thus cultivated or made in the bioreactor and then applied to the hydrocarbon well. In a preferred embodiment, particles of the type described herein, i. e. porous impregnatable particles may be loaded with the products of the bioreactor. The bioreactor, which may be situated at or near the site of the borehole or remote from the borehole, may function to enable the production of any well treatment chemical, such as those described above.

The product isolated from the organisms may be secreted or may be retained in the cell. In the case that the produce is secreted, it may be continuously removed from the cell culture medium, by removing the culture medium and replacing it with the fresh growth medium. The product may then be isolated from the growth medium using standard techniques. Alternatively, the microorganisms may be removed from the bioreactor and the product isolated following cell disruption, using techniques known in the art.

Accordingly, in some embodiments, the present invention provides methods of generating oil or energy substrates comprising delivering a composition comprising transformed hyperthermophilic organisms to an oil bearing formation or other subterranean cavity such as a cave, mine or tunnel via an injection well. In some embodiments, the composition comprising transformed hyperthermophilic organisms further comprises a component selected from the group consisting of energy substrate(s), mineral, salts, vitamins, amino acids, and/or microorganism extracts and combinations thereof. In some embodiments, the methods further comprise delivering a biomass to the oil bearing formation via an oil well. The biomass is preferably selected from the group consisting of sewage, agricultural waste products, brewery grain by-products, food waste, organic industry waste, whey, forestry waste, crops, grass, seaweed, plankton, algae, fish, fish waste, newsprint and combinations thereof. In some preferred embodiments, the biomass is liquefied prior to injection via the oil well. The present invention is not limited to any particular mechanism of action. Indeed, an understanding of the mechanism of action is not necessary to practice the present invention. Nonetheless, it is contemplated that in some embodiments, the transformed hyperthermophilic organisms introduced into an oil bearing formation proliferate and produce acetic acid. The acetic acids makes the rocks of the oil bearing formation more porous thus allowing the recovery of additional oil in the formation. It is contemplated that delivery of additional energy substrates such as biomass will accelerate this process. It is further contemplated that in some embodiments, the oil bearing formation is geothermally heated to a temperature conducive to the growth of transformed hyperthermophilic organisms. Thus, the oil bearing formation can be utilized as a reactor of the production of energy substrates from the degradation of biomass by transformed hyperthermophilic organisms as described above in detail. In these embodiments, hydrogen, ethanol, and/or methane are recovered via wells or pipes inserted into the oil bearing formation into which transformed hyperthermophilic organisms and biomass have been introduced.

### Experimental

### Example 1

### Materials and Methods

Strains and growth conditions. P. furiosus was cultivated under anaerobic conditions at 85° C. in nutrient-rich medium based on ½ SME-medium and supplemented with different organic substrates (8). ½ SME-starch medium contained 0.1% each starch, yeast extract and peptone. For ½ SME-pyruvate medium, the starch was replaced with 40 mM Na-pyruvate. Gelrite (1%) was added for solidification of medium. The antibiotic simvastatin (Toronto Research Inc., Toronto, Canada) was dissolved in ethanol and sterilized by filtration.

General DNA manipulation. Escherichia coli strain DH5α, used for vector construction and propagation, was cultivated at 37° C. in Luria-Bertani (LB) medium. When needed, 100 μg/ml ampicillin was added to media. The vector pYS2 was provided by Prof. Gaël Erauso (Université de la Méditerranée, Marseille, France). Restriction and modification enzymes were purchased from NEB (Ipswich, USA). Plasmid DNA and DNA fragments from agarose gels were isolated using a plasmid mini or gel extraction kit from Qiagen (Hilden, Germany). Phusion High-Fidelity DNA polymerase from Finnzymes (Keilaranta, Finland) was used as a polymerase for PCR. DNA sequencing was performed by Geneart (Regensburg, Germany). Genomic DNA from P. furiosus wild-type and transformed strains was isolated using a DNeasy Blood & Tissue kit from Qiagen.

Construction of the shuttle vectors pYS3 and pYS4. The overexpression cassette for the HMG-CoA reductase gene from P. furiosus was constructed by replacing the native promoter with the strong promoter region (−250 to −1) of the glutamate dehydrogenase gene (gdh; (10)). The fusion of this promoter to the coding region of the HMG-CoA reductase (Primers: PF1848F 5′-ATGGAAATAGAGGAGATTATAGAG-3′ (SEQ ID NO: 3) and PF1848BamHI 5′-ATCATCGGATCCTCATCTCCCAAGCATTTTATGAGC-3′ (SEQ ID NO: 4)) was done by PCR with overhanging ends at the reverse primer for the gdh promoter region gdhPromR-PF1848 (5′-CTCCTCTATTTCCATGTTCATCCCTCCAAATTAGGTG-3′ (SEQ ID NO: 5)). As forward primer for the amplification gdhPromFBamHI (5′GGAACCGGATCCTTGA AAATGGAGTGAGCTGAG-3′ (SEQ ID NO: 6)) was used. The cassette was inserted into the pYS2 vector by replacing the BamHI fragment containing pyrE by the hmg-Co reductase gene. The created vector pYS3 was sequenced and used later for transformation and further modification.

To obtain shuttle vector pYS4, RNA polymerase subunit D (rpoD) was linked to the the fructose-1, 6 bisphosphatase (fbp) promoter and inserted into pYS3. A His₆ tag was attached at the C-terminus of subunit D in addition. The promoter sequence of the fbp was amplified from genomic DNA using the primers EcoRV-PF0613Pr-F (5′-CTATTAGATATCT CCTTAACATTTCTCCAAA-3′ (SEQ ID NO: 7)) and PF0613Prom-R (5′-CTGAACTTCAATTCCGGCCATTTTTTCACCTCCAGAAT-3′ (SEQ ID NO: 8)). Via the PF0613Prom-R primer the promoter sequence had a 3′ overhang for the fusion with the coding region of rpoD. For the amplification of subunit D the primers PF1647-F (5′-AAATGGCC GGAATTGAAGTTCAGATTCTTGA-3′ (SEQ ID NO: 9)) and PF1647-His-R (5′-GTGATGGTGATGGTGATGAGAGGTCAATTTTTGAAGTTCAC-3′ (SEQ ID NO: 10)) were used. This step introduced the incorporation of the sequence for the His₆ tag at the C-terminus of rpoD. The rpoD-His₆ was fused with the terminating region of the histone A1 gene of P. furiosus. (24). The primer pair His-PF1831Term-F (5′-CATCACCATCACCATCACTGAAATCTTT TTTAGCACTT-3′ (SEQ ID NO: 11)) and PF1831T-EcoRV-R (5′-TCAATTGATATCA CCCTAGAAAAAGATAAGC-3′ (SEQ ID NO: 12)) created the terminating region of the histone A1 gene with a part overlapping the rpoD-His₆ at the 5′-end that was used to fuse the fragments by PCR. Finally, the construct was integrated into the pYS3 vector next to the hmg-CoA reductase cassette using the flanking EcoRV sites. The construction of the plasmid was verified by DNA sequencing.

Transformation of P. furiosus. P. furiosus cultures grown at 75° C. to a cell density between 0.8-1.0×10⁸ per ml were used for transformation. For a transformation reaction the cells of 3 ml grown culture were collected anaerobically by centrifugation (10 minutes at 6000 g) and resuspended in a total volume of 100 μl transformation solution containing ½ SME (without KH₂PO₄), 40 mM Na-pyruvate, 4.7 mM NH₄Cl and 80 mM CaCl₂. The pH was adjusted to 7.0 with HCl. Cells were incubated at 4° C. for 90 minutes under anaerobic conditions. After 30 minutes 0.5 pmol pYS3 or pYS4 were added. After a heat shock at 80° C. for 3 minutes, the cells were again incubated for 10 min at 4° C. and then cultivated in ½ SME-starch liquid medium in the presence of 10 μM simvastatin at 85° C. for 48 h. Later, the cells were plated on ½ SME medium with starch as substrate and containing 10 μM simvastatin. The plates were incubated at 85° C. for 48 h.

Growth properties of P. furiosus and P. furiosus pYS3 and pYS4 transformants. To analyze resistance toward simvastatin, pYS3 transformed cells were cultivated in ½ SME-starch medium supplemented with 1, 5, 10, or 20 μM simvastatin at 85° C. Wild-type P. furiosus cells were also cultivated in ½ SME-starch medium at 85° C., but without simvastatin. Cell densities were measured at appropriate intervals. Cell counts were analyzed with a Thoma counting chamber (0.02-mm depth; Marienfeld, Lauda-Königshofen, Germany) under a phase-contrast microscope. To determine the expression of subunit D under glycolytic or gluconeogenetic conditions, P. furiosus pYS4 cells were grown either in ½ SME-starch or ½ SME-pyruvate medium in the presence of 10 μM simvastatin at 85° C.

Detection of RpoD and RpoD-His₆ by western blot analysis. For the preparation of cell extracts 10 g of P. furiosus wild-type or P. furiosus cells transformed with pYS4 were resuspended in 30 ml buffer (40 mM HEPES, 500 mM NaCl, 10 mM imidazole, 15% glycerol, pH 7.5), sonicated on ice and treated with glass beads using a FastPrep-24 (M. P. Biomedicals, Irvine, USA) for complete cell lysis. After centrifugation (100.000 g for 1 h at 4° C.) the protein concentrations of the clarified supernatants were determined by Bradford assays. For quantification of the expression levels of RpoD or RpoD-His₆ western blots were done as previously described using polyclonal antibodies raised against recombinant subunits A″ or D from P. furiosus (9). The signals were visualized using a Cy5-labelled secondary anti-rabbit antibody from Thermoscientific (Waltham, USA) and a fluorescence image analyzer (FLA-5000, Fuji, Japan).

Purification of RpoD-His₆ and RNAP-His₆. The cell extracts prepared as described in the previous section were applied onto 1-ml Ni²⁺-charged HisTrap HP columns (GE Healthcare). Bound proteins were eluted in one step using an elution buffer containing 300 mM imidazole instead of 10 mM. To separate free RpoD-His₆ from the fraction incorporated into the RNAP the eluate was loaded onto a Superdex 200 gel filtration column (GE Healthcare) equilibrated with 40 mM HEPES, pH 7.3, 250 mM KCl, 2.5 mM MgCl₂, 0.5 mM EDTA, 20% glycerol. Aliquots of the fractions were analyzed for RNAP activity using a specific in vitro transcription assay (10) and SDS-PAGE analysis.

Southern blot analysis. Total genomic DNA was digested with EcoRV and the resulting restriction fragments were separated on a 1% agarose gel. After electrophoresis, the DNA was transferred to a nylon membrane (Roche Applied Science, Mannheim, Germany) by capillary blot. A part of the rpoD gene was amplified by PCR using the primer pair RpoD500-F (5′-CCAACATTTGCAGTTGATGAAG-3′ (SEQ ID NO: 13)) and RpoD500-R (5′-CTCTTCGAAATCCTTTGGTATGTAG-3′ (SEQ ID NO: 14)). This segment was used as probe to detect the RNAP subunit D gene in genomic and in plasmid DNA. The labelled probe was generated by the random primed method using the NEBlot kit (NEB, Ipswich, USA) in the presence of digoxigenin-11-dUTP (Roche Applied Science, Mannheim, Germany). After hybridisation the signals were detected using anti-digoxigenin antibodies conjugated with alkaline phosphatase according to the instructions of the producer (Roche Applied Science, Mannheim, Germany).

### Results

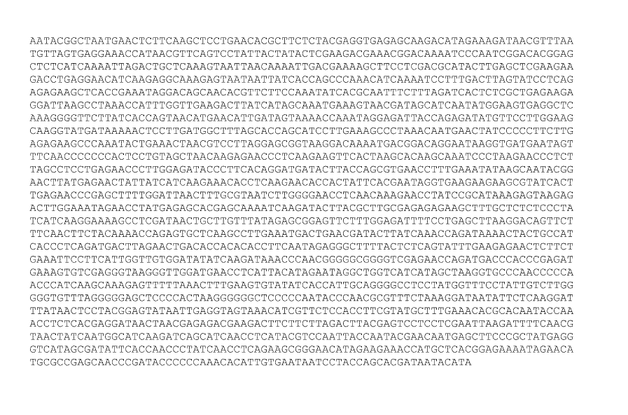

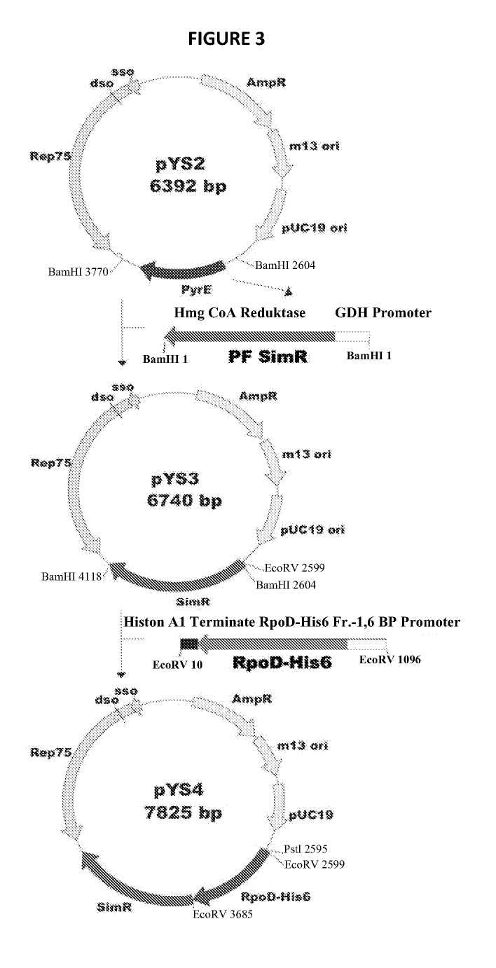

Transformation in P. furiosus with a redesigned shuttle vector of pYS2. The selection mechanism of the shuttle vector pYS2 is based on a uracil auxotrophic strain of P. abyssi which has a mutation in the pyrE gene (17). The plasmid contains a wild-type copy of the pyrE gene of S. acidocaldarius and successful transformation complements the uracil auxotrophy. As our attempts to construct a uracil auxotrophic strain of P. furiosus were not successful (data not shown) we redesigned the vector pYS2. In the new construct pYS3 the pyrE gene was substituted by the hmg-CoA reductase gene and for an efficient expression this gene was fused with the strong gdh promoter from P. furiosus ((10); FIG. 3).

As overexpression of the HMG-CoA reductase led to the resistance against simvastatin in T. kodokaraensis (18) we also analyzed the effect of various concentrations of simvastatin on the growth of P. furiosus. In ½ SME-pyruvate medium supplemented with 5, 10, or 20 μM simvastatin, growth was inhibited for only one day, if the cells were incubated at 95° C. In contrast, incubation at 85° C. with similar concentrations prevented growth for three days. This indicates that the stability of simvastatin is dramatically decreased at 95° C., but 85° C. seems to be an appropriate temperature for selection of transformants. This reduced incubation temperature still allows growth of P. furiosus in a reasonable time in liquid as well as in solidified medium. In the first experiments, the new construct pYS3 was used to transform P. furiosus according to the published CaCl₂ procedure for T. kodakaraensis with some minor modifications (23): The heat shock was performed for 3 minutes at 80° C. instead of 45 seconds at 85° C. and cells were incubated in the cold at 4° C. instead at 0° C. Transformants were selected by growing cells for 48 h in liquid medium in the presence of 10 μM simvastatin. Growth was only observed when cells were transformed with plasmid pYS3 and not when cells were treated in control reactions with transformation solution not containing the plasmid. The transformation efficiency in liquid medium was approximately 5×10² transformants per μg pYS3 plasmid DNA. For the isolation of single transformants cells grown in liquid cultures were plated on culture medium containing 10 μM simvastatin. The plating efficiency of the transformants in the presence of 10 μM simvastatin was ˜15% (the plating efficiency of WT cells on media not containing the antibiotics was ˜78%).

A few simvastatin resistant colonies were selected and further analyzed for the presence of the plasmid by PCR amplification. To provide evidence that the plasmid was stably replicated in Pyrococcus the plasmid was isolated again from Pyrococcus after several transfers (4-5 times) of cells in fresh culture medium. Using this isolated plasmid DNA it was possible to successfully re-transform E. coli (data not shown). This clearly demonstrates that this redesigned shuttle vector including the plasmid pGTS from P. abyssi was also stably replicated as an external DNA element in P. furiosus.

Induced expression of subunit D of the RNAP. As next step it was analyzed whether the shuttle vector could be converted into an expression vector which allows the expression of proteins under the control of a regulated promoter. Subunit D of the archaeal RNAP was used as a model protein and an additional copy of this gene was inserted into the shuttle vector under the control of the fructose-1-6 bisphosphatase (PF0613) promoter (FIG. 3, pYS4). To allow a simple and rapid purification of the protein a His₆ tag at the C-terminus was introduced and for efficient termination of transcription the terminator from the histone gene hpyA1 was linked to the 3′-end of the gene (24). The PF0613 promoter is repressed under glycolytic and induced under gluconeogenetic conditions (13, 16).

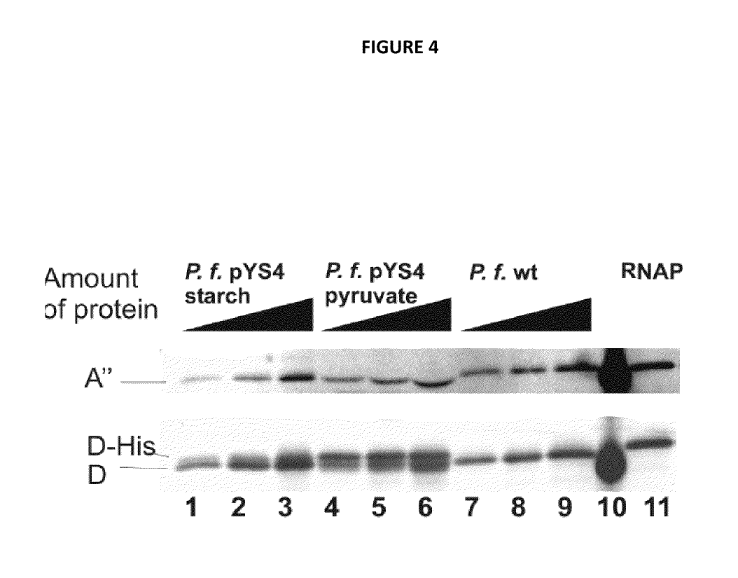

The new construct pYS4 was transformed into Pyrococcus and a single colony was transferred into liquid medium and first cultivated under glycolytic conditions in the presence of starch. Later, the same culture was transferred to gluconeogenetic conditions using a medium containing pyruvate as energy source. In each case the expression of subunit D was analyzed in crude extracts and compared with the wild-type by a western blot assay using antibodies against RNAP subunit D (FIG. 4). Identical amounts of RNAP were applied to gels used for western blots as shown by immunostaining using the antibody raised against RNAP subunit A″. Analysis of the crude extracts of the wild-type strain revealed only one signal corresponding to subunit D (FIG. 4, lanes 7-9). In contrast, the crude extracts of the transformants grown with starch (lanes 1-3) or pyruvate (lanes 4-6) contained an additional polypeptide migrating slightly slower than wild-type subunit D. This signal corresponding to the additional copy of subunit D encoded on the plasmid differed in size due to the existence of the His₆ tag at the C-terminus. The additional signal found in transformants was rather weak in cells grown with starch and much stronger in cells grown with pyruvate (FIG. 4, compare lanes 1-3 with 4-6). This clearly demonstrates that the promoter of the additional subunit D copy on the plasmid is strongly induced under gluconeogenetic conditions and therefore this system is useful for regulated expression of proteins in P. furiosus.

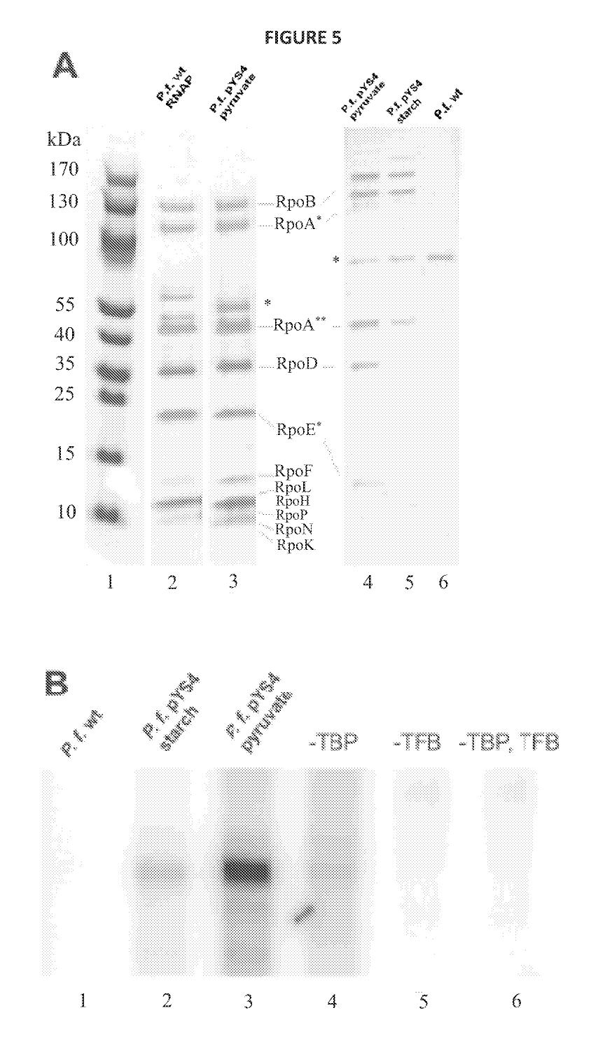

Purification of archaeal RNAP by immobilized metal ion affinity chromatography. To analyze whether this modified subunit D containing a His₆ tag at the C-terminus also assembles into the archaeal RNAP, the crude extract of cells transformed with pYS4 was applied onto Ni-NTA columns. Specific bound proteins were eluted with a buffer containing 300 mM imidazole. To separate subunit D assembled into the archaeal RNAP from the free polypeptide the pooled subunit D-containing fractions from the Ni-NTA column were further purified by gel filtration chromatography. The RNAP containing fraction isolated by this two-step procedure from the transformant grown with pyruvate was compared with conventionally purified native RNAP (19) by gradient SDS PAGE and silver staining (FIG. 5A, lanes 2 and 3). Both RNAPs showed an almost identical pattern. This indicates that the overexpressed subunit D with the His₆ tag assembles into the RNAP and the whole enzyme can be isolated by immobilized metal ion affinity chromatography.

Starting with similar amounts of cells for purification, five-times more RNAP was isolated from transformant cells grown with pyruvate compared to cells grown with starch (FIG. 3A, compare lanes 4 and 5). As expected, RNAP was not enriched in similar fractions purified from extracts of the wild-type strain by the same procedure (FIG. 5A, lane 6). The band labelled with an asterisk was purified from wild-type cells as well as from the transformants after Ni-NTA affinity chromatography and Superdex 200 gel filtration. This multimeric polypeptide was not characterized in any detail here.

To check whether the affinity purified RNAP fractions (FIG. 5A, lanes 4 to 6) are functionally active these fractions were analyzed by in vitro transcription experiments. The affinity purified fractions were able to synthesize run-off RNA products from the gdh promoter in the presence of both archaeal transcription factors, TBP and TFB ((10); FIG. 5B, lanes 2 and 3). When one transcription factor or both were omitted, transcription was abolished (FIG. 5B, lanes 4 to 6). Taken together, these data indicate that subunit D with a C-terminal His₆ tag assembles into the RNAP. As expected the amount of RNAP containing His6-tagged D that can be purified from a given amount of cells is higher when subunit D was overexpressed from the gluconeogenetic promoter. Furthermore, it is possible to specifically purify this fraction of the RNAP by Ni-NTA and size exclusion chromatography from the crude extract. The purified fraction is functionally active and not contaminated with TBP and TFB. As expected, this procedure did not allow the purification of RNAP without the C-terminal His₆ tag at subunit D (FIG. 5B, lane 1).

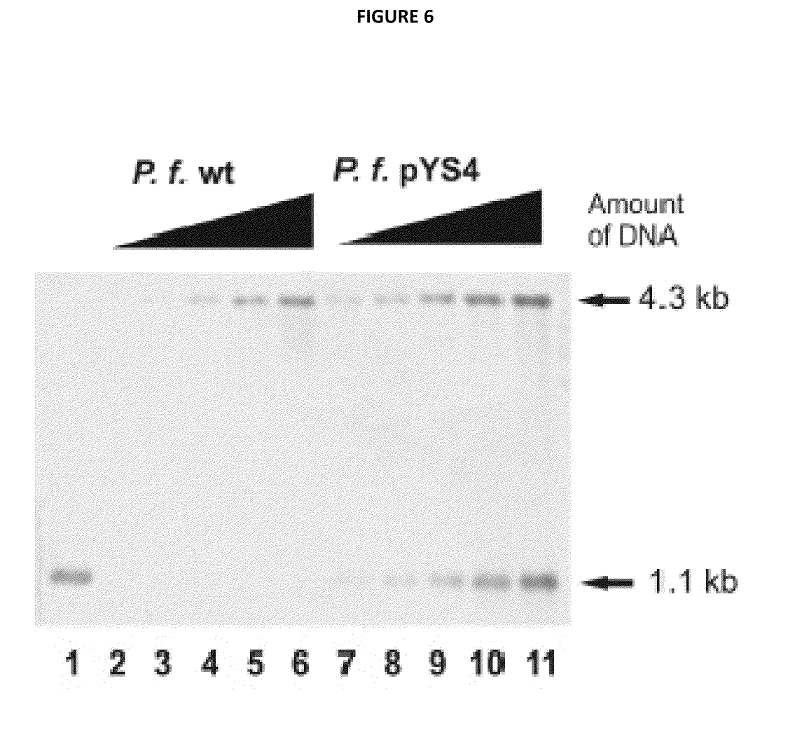

Copy number of pYS4 in P. furiosus. To determine the copy number of this shuttle vector in P. furiosus EcoRV-digested total DNA was analyzed by Southern blot experiments. The DNA sequence of subunit D was used as probe. When wild-type P. furiosus DNA was analyzed a 4.3 kb signal was identified. This exactly corresponds to the predicted size of a fragment containing subunit D in chromosomal DNA restricted with EcoRV (FIG. 6, lanes 2-6). When transformants were analyzed beside the genomic fragment an additional band with a size of 1.1 kb was observed (FIG. 6, lanes 7 to 11). As this signal was also present in the control lane with EcoRV-hydrolyzed plasmid DNA (lane 1) these results clearly demonstrate the presence of plasmid pYS4 in transformed cells. The finding that the strength of both signals arising from the genomic fragment and from the plasmid are in a similar range indicates that the copy number of the plasmid is approximately in the same ratio as the number of chromosomes of one cell. To exclude the possibility that the ratio between plasmid and chromosomal DNA was changed during the DNA purification procedure, the ratio between plasmid and chromosomal DNA was analyzed in crude extracts in addition. This experiment confirmed the results that the copy number of the plasmid pYS4 in P. furiosus was between one and two (data not shown).

### Discussion

The present invention provides a shuttle expression vector system for P. furious and E. coli allowing the regulated expression of proteins in Pyrococcus. A published shuttle vector of P. abyssi has been redesigned using the overexpression of the HMG-CoA reductase as a selection marker which confers resistance to the antibiotic simvastatin as described earlier for T. kodakaraensis (18). The copy number of the new vectors pYS3 and pYS4 was dramatically reduced in comparison to the pYS2 shuttle vector used in P. abyssi. The copy number of the shuttle vector pYS2 was 20 to 30 copies per chromosome and was therefore in the same range as described for the wild-type pGT5 plasmid from P. abyssi (6, 17). At present we have no explanation for the dramatic reduction of the copy number to one or two per chromosome in P. furious. As in our construct the transcription of the hmg-CoA reductase gene occurs in opposite direction to the replication of the plasmid, we have also analyzed whether insertion of the hmg-CoA reductase gene in the opposite direction affects the copy number. First experiments indicate that the transcriptional orientation of the hmg-CoA reductase gene does not influence the copy number of the plasmid (data not shown). We assume that the maintenance of this plasmid in P. furiosus is mainly driven by the antibiotic resistance and the mechanism responsible to maintain a certain copy number in P. abyssi is absent in P. furiosus. A reduced copy number in a different host was also observed when plasmids pAG1 and pAG2 from P. abyssi were transferred to P. furiosus (1) or when a plasmid from Thermococcus nautilus was transferred to T. kodakaraensis (21).

Although the copy number of the shuttle vector described in this paper is low, the possibility to transform P. furiosus and to use this shuttle vector for the regulated expression of plasmid-encoded genes now allows the development of a genetic system. Our recent results suggest that it will be also possible to mutate the chromosome of P. furiosus using the overexpression of the HMG-CoA reductase as a selection marker against simvastatin (data not shown). But also the shuttle vector based regulated expression system offers novel intriguing possibilities for future developments. As we could successfully demonstrate that this system allows overexpression of a His₆ tagged subunit D under the control of a gluconeogenetic promoter whose expression was dependent upon the substrate in the growth medium, the system described here can be used for production of recombinant proteins in P. furiosus. It could be an alternative system for expression of proteins which are difficult to produce in E. coli, especially for proteins of hyperthermophiles which might have a low propensity to fold properly at low temperature like the Sor (sulfur oxygenase/reductase) protein from Acidianus ambivalens which was produced with higher efficiency in a hyperthermophilic Sulfolobus expression system than in E. coli (2).

We provide also evidence that this system can be used to isolate an active fraction of RNAP in a two step purification procedure when a His₆ tagged additional copy of subunit D was overexpressed in Pyrococcus cells. This system allows to overexpress mutant subunits in Pyrococcus and to isolate the RNAP containing mutations for structure function analyses as described for T. kodakaraensis (11, 21). Using a low level of expression of a particular subunit from the PF0613 promoter (growth on starch) it should be also possible to introduce point mutations into functional important regions of this subunit. Therefore, this system is a perfect complementation of our previously described system for the reconstitution of the 11-subunit RNAP from individual subunits in vitro (19). Furthermore, this system will be useful for the construction of a reporter gene assay, which should allow a rapid in vivo analysis of promoter sequences or of regulatory DNA elements in P. furiosus. Taken together, the presented shuttle vector based transformation system for P. furiosus is an important first step to establish a complete genetic toolbar for one of the hyperthermophilic key organisms in archaeal research for analysis of recombination (27), replication (5, 14), transcription (25) and metabolism (12).

### Example 2

### Genetic Engineering of the Chromosomal rpoD Gene of the RNA polymerase from Pyrococcus furiosus

This example provides a demonstration that the selectable marker can be used not only for the selection of plasmids but also for the selection of chromosomal mutants. Using the marker, a Strep-His-Tag was introduced via double-crossover recombination at the C-terminus of subunit D of the RNA polymerase in Pyrococcus furiosus. Active RNA polymerase was purified from this mutant strain in a two step procedure consisting of Ni-NTA and gel filtration chromatography.

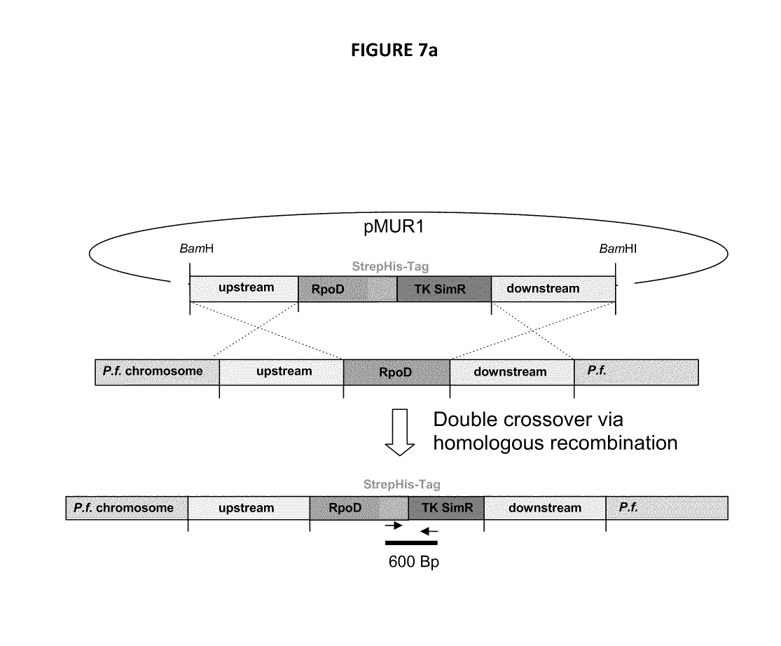

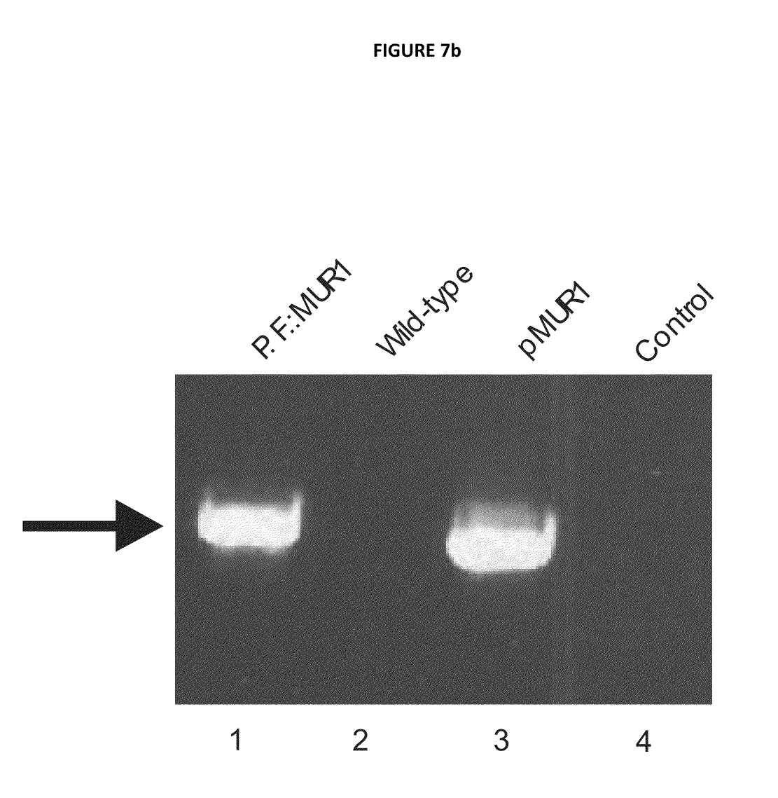

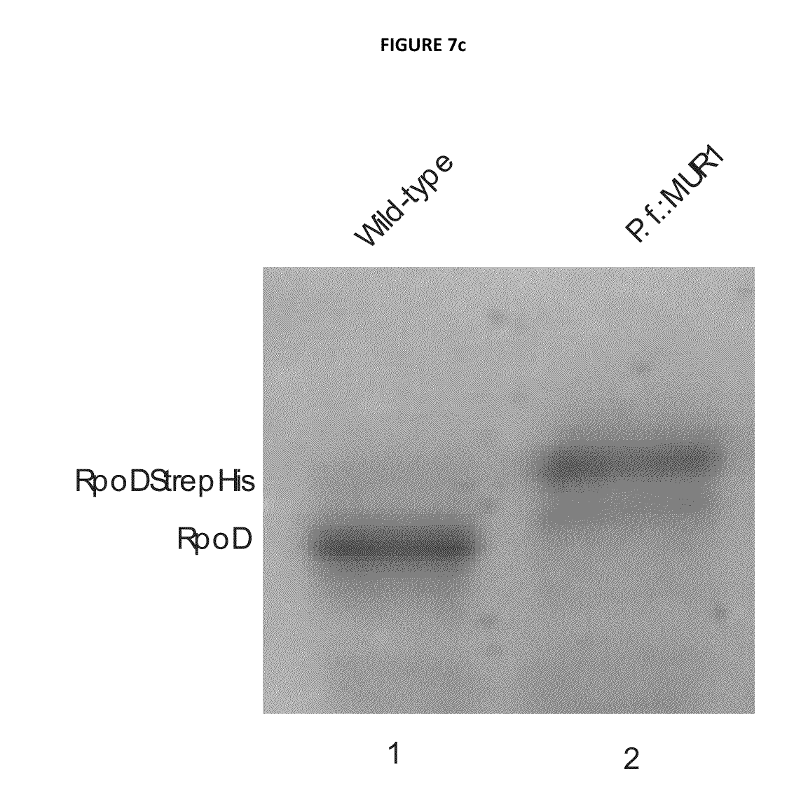

FIG. 7a provides a schematic drawing of pMUR1, a plasmid designed for the introduction of a C-terminal Strep-His-Tag into subunit rpoD. The homologous up- and downstream regions promoting double crossover are shown in identical colours. Linearized plasmid pMUR1 was used to transform wild-type Pyrococcus furiosus as described according to a previously published protocol (26).

FIG. 7b provides the results of a PCR analysis of the rpoD gene locus. Primers corresponding to the Strep-His-Tag and to the simvastatin resistence cassette (indicated in FIG. 1A) were used to confirm gene modification of rpoD. The 600 by fragment amplified from genomic DNA of the transformant indicates successful recombination (lane 1). As expected this primer pair allows no amplification using wild-type DNA (lane 2). Lane 3 and 4 are additional control lanes with and without the corresponding template DNAs.

FIG. 7c provides the results of a Western Blot analysis of the modified subunit RpoD. Crude extract from wild-type and from the transformant P.f.:MUR1 was analyzed by a western blot using an anti-subunit D antibody. Due to the introduction of the StrepHis-Tag at the C-terminus of subunit D the signal of Rpo D migrates slower than the corresponding wild-type signal of the transformant.

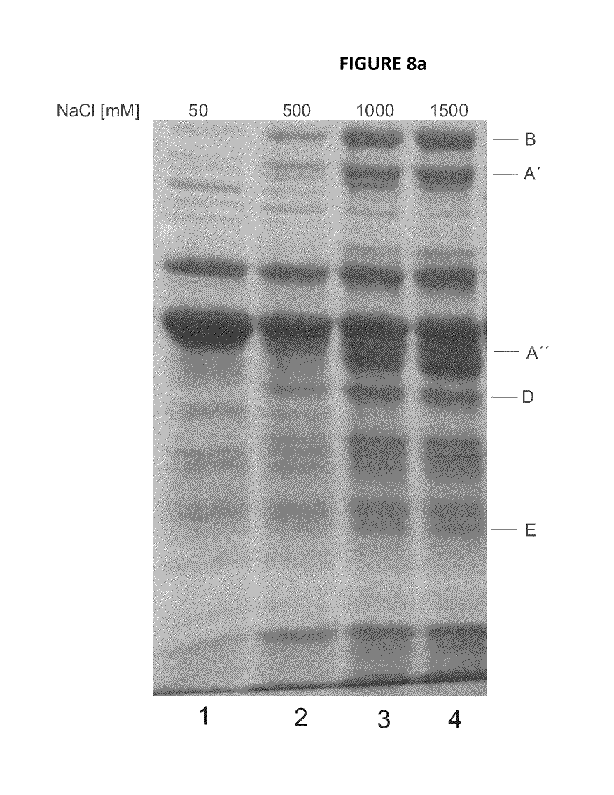

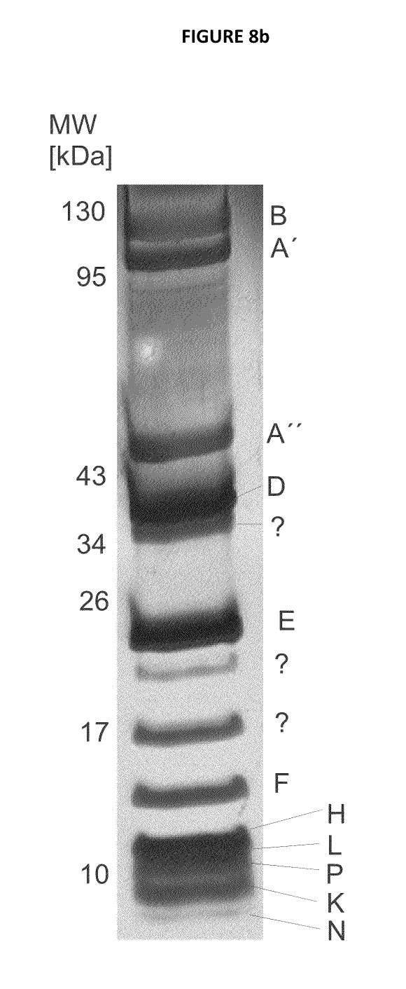

FIG. 8a provides results of Ni-NTA chromatography with cell extracts containing different NaCl concentrations. To optimize the purification protocol for the RNA polymerase, extracts with loading buffer containing NaCl concentrations from 50 (lane 1), 500 (lane 2), 1000 (lane 3) and 1500 mM (lane 4) were applied to Ni-NTA columns. With increasing NaCl concentrations the amount of RNA polymerase binding to the Ni-NTA column was improved. Elution of bound proteins was performed as described previously (26)

FIG. 8b provides a silver stained SDS gel of the purified RNA polymerase after Superdex 200 chromatography. The corresponding subunits of the RNA polymerase are labelled. After the two step purification procedure the RNA polymerase contains only three additional proteins labelled with a “?”. The identification of these proteins is in progress.

The experiments deomonstrate that: A. Using the simvastatin resistence marker for the selection of transformants we could successfully introduce for the first time a modified gene into the chromosomal DNA from Pyrococcus furiosus; B. The introduction of a His-Strep-Tag at the C-terminus of subunit D simplified the purification of the enzyme dramatically. It can be now purified by a two step procedure; and C. Using this procedure we can isolate 10 mg RNA polymerase from 20 g cells (wet weight).

### REFERENCES

1. Aagaard, C., I. Leviev, R. N. Aravalli, P. Forterre, D. Prieur, and R. A. Garrett. 1996. General vectors for archaeal hyperthermophiles: strategies based on a mobile intron and a plasmid. FEMS Microbiol. Rev. 18:93-104.
2. Albers, S. V., M. Jonuscheit, S. Dinkelaker, T. Urich, A. Kletzin, R. Tampe, A. J. Driessen, and C. Schleper. 2006. Production of recombinant and tagged proteins in the hyperthermophilic archaeon Sulfolobus solfataricus. Appl. Environ. Microbiol. 72:102-111.
3. Aravalli, R. N., and R. A. Garrett. 1997. Shuttle vectors for hyperthermophilic archaea. Extremophiles 1:183-191.
4. Atomi, H., and T. Imanaka. 2008. Targeted gene disruption as a tool for establishing gene function in hyperthermophilic Archaea, p. 213-223. In F. Robb, G. Antranikian, D. Grogan, and A. Driessen (ed.), Thermophiles: Biology and Technology at High Temperatures, CRC Press, Boca Raton, Fla.
5. Emptage, K., R. O'Neill, A. Solovyova, and B. A. Connolly. 2008. Interplay between DNA polymerase and proliferating cell nuclear antigen switches off base excision repair of uracil and hypoxanthine during replication in archaea. J. Mol. Biol. 383:762-771.
6. Erauso, G., F. Charbonnier, T. Barbeyron, P. Forterre, and D. Prieur. 1992. Preliminary characterization of a hyperthermophilic archaebacterium with a plasmid, isolated from a north Fiji basin hydrothermal vent. C. R. Acad., Sci. 314:387-393.
7. Erauso, G., S. Marsin, N. Benbouzid-Rollet, M. F. Baucher, T. Barbeyron, Y. Zivanovic, D. Prieur, and P. Forterre. 1996. Sequence of plasmid pGT5 from the archaeon Pyrococcus abyssi: evidence for rolling-circle replication in a hyperthermophile. J. Bacteriol. 178:3232-3237.
8. Fiala, G., and K. O. Stetter. 1986. Pyrococcus furiosus sp. Nov. represents a novel genus of marine heterotrophic archaebacteria growing optimally at 100° C. Arch. Microbiol. 145:56-61.
9. Hausner, W., and M. Thomm. 1995. The translation product of the presumptive Thermococcus celer TATA-binding protein sequence is a transcription factor related in structure and function to Methanococcus transcription factor B. J. Biol. Chem. 270:17649-17651.
10. Hethke, C., A. C. Geerling, W. Hausner, W. M. de Vos, and M. Thomm. 1996. A cell-free transcription system for the hyperthermophilic archaeon Pyrococcus furiosus. Nucleic Acids Res. 24:2369-2376.
11. Hirata, A., T. Kanai, T. J. Santangelo, M. Tajiri, K. Manabe, J. N. Reeve, T. Imanaka, and K. S. Murakami. 2008. Archaeal RNA polymerase subunits E and F are not required for transcription in vitro, but a Thermococcus kodakarensis mutant lacking subunit F is temperature-sensitive. Mol. Microbiol. 70:623-633.
12. Jenney, F. E., Jr., and M. W. Adams. 2008. Hydrogenases of the model hyperthermophiles. Ann. N. Y. Acad. Sci. 1125:252-266.
13. Kanai, T., J. Akerboom, S. Takedomi, H. J. van de Werken, F. Blombach, J. van der Oost, T. Murakami, H. Atomi, and T. Imanaka. 2007. A global transcriptional regulator in Thermococcus kodakaraensis controls the expression levels of both glycolytic and gluconeogenic enzyme-encoding genes. J. Biol. Chem. 282:33659-33670.
14. Kiyonari, S., S. Tahara, M. Uchimura, T. Shirai, S. Ishino, and Y. Ishino. 2009. Studies on the base excision repair (BER) complex in Pyrococcus furiosus. Biochem. Soc. Trans. 37:79-82.
15. Lam, W. L., and W. F. Doolittle. 1992. Mevinolin-resistant mutations identify a promoter and the gene for a eukaryote-like 3-hydroxy-3-methylglutaryl-coenzyme A reductase in the archaebacterium Haloferax volcanii. J Biol Chem 267:5829-5834.
16. Lee, S. J., M. Surma, W. Hausner, M. Thomm, and W. Boos. 2008. The role of TrmB and TrmB-like transcriptional regulators for sugar transport and metabolism in the hyperthermophilic archaeon Pyrococcus furiosus. Arch. Microbiol. 190:247-256.
17. Lucas, S., L. Toffin, Y. Zivanovic, D. Charlier, H. Moussard, P. Forterre, D. Prieur, and G. Erauso. 2002. Construction of a shuttle vector for, and spheroplast transformation of, the hyperthermophilic archaeon Pyrococcus abyssi. Appl. Environ. Microbiol. 68:5528-5536.
18. Matsumi, R., K. Manabe, T. Fukui, H. Atomi, and T. Imanaka. 2007. Disruption of a sugar transporter gene cluster in a hyperthermophilic archaeon using a host-marker system based on antibiotic resistance. J. Bacteriol. 189:2683-2691.
19. Naji, S., S. Grunberg, and M. Thomm. 2007. The RPB7 orthologue E′ is required for transcriptional activity of a reconstituted archaeal core enzyme at low temperatures and stimulates open complex formation. J. Biol. Chem. 282:11047-11057.
20. Santangelo, T. J., L. Cubonova, C. L. James, and J. N. Reeve. 2007. TFB1 or TFB2 is sufficient for Thermococcus kodakaraensis viability and for basal transcription in vitro. J. Mol. Biol. 367:344-357.
21. Santangelo, T. J., L. Cubonova, and J. N. Reeve. 2008. Shuttle vector expression in Thermococcus kodakaraensis: contributions of cis elements to protein synthesis in a hyperthermophilic archaeon. Appl. Environ. Microbiol. 74:3099-3104.
22. Sato, T., T. Fukui, H. Atomi, and T. Imanaka. 2005. Improved and versatile transformation system allowing multiple genetic manipulations of the hyperthermophilic archaeon Thermococcus kodakaraensis. Appl. Environ. Microbiol. 71:3889-3899.
23. Sato, T., T. Fukui, H. Atomi, and T. Imanaka. 2003. Targeted gene disruption by homologous recombination in the hyperthermophilic archaeon Thermococcus kodakaraensis KOD1. J. Bacteriol. 185:210-2020.
24. Spitalny, P., and M. Thomm. 2008. A polymerase III-like reinitiation mechanism is operating in regulation of histone expression in archaea. Mol. Microbiol. 67:958-970.
25. Thomm, M., C. Reich, S. Grunberg, and S. Naji. 2009. Mutational studies of archaeal RNA polymerase and analysis of hybrid RNA polymerases. Biochem. Soc. Trans. 37:18-22.
26. Waege, I., G. Schmid, S. Thumann, M. Thomm , and W. Hausner. 2010. Shuttle vector-based transformation system for Pyrococcus furiosus. App.. Environ. Microbiol. 76:3306-3313.
27. Williams, R. S., G. Moncalian, J. S. Williams, Y. Yamada, O. Limbo, D. S. Shin, L. M. Groocock, D. Cahill, C. Hitomi, G. Guenther, D. Moiani, J. P. Carney, P. Russell, and J. A. Tainer. 2008. Mre11 dimers coordinate DNA end bridging and nuclease processing in double-strand-break repair. Cell 135:97-109.

What is claimed is:

1. A shuttle vector comprising, in operable association:
a bacterial replication origin, a bacterial selection marker gene, a rolling circle replication initiator protein gene, an antibiotic resistance gene that confers selectability in a archaeaon, and a regulated archaeal promoter 5′ to a cloning site.

2. The shuttle vector of claim 1, wherein said rolling circle replication initiator protein gene is selected from the group consisting of Rep74 and Rep75.

3. The shuttle vector of claim 1, wherein said antibiotic resistance gene that confers selectability in an archaeon is hmg-CoA reductase.

4. The shuttle vector of claim 1, wherein said bacterial replication origin is oriC.

5. The shuttle vector of claim 1, wherein said bacterial selection marker gene is different than said antibiotic resistance gene that confers selectability in an archaeon.

6. The shuttle vector of claim 1, wherein said bacterial selection marker gene is selected from the group consisting of an auxotrophic marker gene and a gene that confers antibiotic resisstance on a host cell.

7. The shuttle vector of claim 6, wherein said antibiotic selected from the group consisting of kanamycin, ampicillin, tetracycline, Zeocin, neomycin, chloramphenicol and hygromycin.

8. The shuttle vector of claim 6, wherein said auxotrophic marker is a gene selected from the group consisting of LEU2 gene, HIS3 gene, TRP1 gene, URA3 gene, ADE2 gene and LYS2 gene.

9. The shuttle vector of claim 1, further comprising a gene of interest in operable association with said archaeal promoter.

10. The shuttle vector of claim 9, wherein said gene of interest is inserted at said cloning site.

11. The shuttle vector of claim 9, wherein said gene of interest is an enzyme.

12. The shuttle vector of claim 1, wherein said vector is pYS3.

13. The shuttle vector of claim 12, wherein said vector is encoded by SEQ ID NO: 1.

14. A host cell comprising the vector of claim 1.

15. The host cell of claim 14, wherein said host cell is selected from the group consisting of members of the genera Thermococcus and Pyrococcus.

16. A method of expressing a gene of interest in an archaea comprising:
culturing an archaeon comprising the shuttle vector of claim 9 under conditions suitable for expression of said gene of interest from said promoter.

17. A method of producing a protein of interest encoded by a gene of interest in an archaea comprising:
culturing an archaeon comprising the shuttle vector of claim 9 under conditions suitable for expression of said protein of interest from said gene of interest.

18. The method of claim 17, further comprising purifying said protein of interest.

19. A method for transforming an archaeon comprising:
providing a shuttle vector according to claim 1 and introducing said shuttle vector into an archaeon.

20. A process for producing an energy substrate from a biomass comprising:
contacting a biomass with an archaeon transformed with a vector according to claim 9.

21. A method of screening for altered protein function comprising:
mutating a nucleic acid encoding a protein of interest;
transforming an archaeon with said nucleic acid;
screening said archaeon for expression a protein of interest with a desired property.

22. The method of claim 21, wherein said mutating comprises a method selected from the group consisting of error prone PCR, chemical mutagenesis, and gene shuffling.

23. The method of claim 21, wherein said desired property is selected form the group consisting of enhanced thermostability and enhanced action on a desired substrate.

24. The method of claim 21, further comprising the step of selecting and isolating said archaea expressing a protein of interest with a desired property.

25. The method of claim 21, wherein multiple mutations are introduced into said nucleic acid of interest.

26. The method of claim 21, wherein greater than 100,000 transformed archaea are screened.

27. A method of genetically altering an archaeon comprising:
transforming said archaea with a shuttle vector comprising nucleic acid sequences that are homologous to the target gene of interest, wherein said homologous sequences flank a selectable marker.

28. The method of claim 27, further comprising the step of selecting for archaea expressing the selectable marker.

29. The method of claim 27, wherein said target gene of interest is selected from the group consisting of membrane bound hydrogenases and aldehyde ferredoxin oxidoreductase.

30. An archaeon produced by the process of claim 27.

31. An archaeon comprising an exogenous gene, wherein said archaea is a Pyrococcus sp.

32. An archaeon comprising a disrupted endogenous gene, wherein said archaeon is a Pyrococcus sp.
